(19) United States
(12) Patent Application Publication
(10) Pub. No.: US 2019/0287674 A1
(43) Pub. Date: Sep. 19, 2019

(54) MEDICAL SIGNAL PROCESSING APPARATUS

(71) Applicant: Canon Medical Systems Corporation, Otawara-shi (JP)

(72) Inventors: Shuhei NITTA, Tokyo (JP); Hidenori TAKESHIMA, Kawasaki (JP)

(73) Assignee: Canon Medical Systems Corporation, Otawara-shi (JP)

(21) Appl. No.: 16/429,771

(22) Filed: Jun. 3, 2019

Related U.S. Application Data
(63) Continuation of application No. PCT/JP2018/044729, filed on Dec. 5, 2018.

(30) Foreign Application Priority Data
Dec. 20, 2017 (JP) 2017-243941
Dec. 4, 2018 (JP) 2018-227295

(51) Int. Cl. G16H 30/40; G06T 7/00; G01R 33/48; G01R 33/54

(52) U.S. Cl. CPC G16H 30/40; G06T 7/0012; G06N 20/00; G01R 33/54; G01R 33/4818

(57) ABSTRACT

According to one embodiment, a medical signal processing apparatus includes processing circuitry. The processing circuitry inputs a medical signal to a learned model configured to output one of the following: a corrected signal that is corrected so as to reduce a pattern of the medical signal, the pattern appearing at a location shifted by a known shift amount in a known direction; pattern-related information relating to the pattern; or disease information relating to the medical signal. The processing circuitry outputs, by using the direction and the shift amount, one of the corrected signal, the pattern-related information, or the disease information.

### CROSS-REFERENCE TO RELATED APPLICATIONS

This application is a Continuation Application of PCT Application No. PCT/JP2018/044729, filed Dec. 5, 2018 and based upon and claiming the benefit of priority from Japanese Patent Applications No. 2017-243941, filed Dec. 20, 2017, and No. 2018-227295, filed Dec. 4, 2018, the entire contents of all of which are incorporated herein by reference.

### FIELD

Embodiments described herein relate generally to a medical signal processing apparatus.

### BACKGROUND

In conventional magnetic resonance imaging, artifacts may be caused in an image after being subjected to Fourier transformation or inverse Fourier transformation due to various factors. For example, when under-sampling is performed to collect data stored in a reduced number of lines in k-space, aliasing may occur in an image. For a further example, if data is collected by an echo planar imaging (EPI) method, artifact called N/2 artifact may occur in an image.

If a convolutional neural network (hereinafter, a CNN) that has been learned so as to reduce artifact is adopted for the image, accuracy in artifact reduction for some input target cannot be improved by a conventional CNN protocol. In other words, for example, if a magnetic resonance image with aliasing, such as N/2 artifact, is used as an input target to the CNN, accuracy in reduction of aliasing may not be improved.

### BRIEF DESCRIPTION OF THE DRAWINGS

FIG. 1 is a diagram showing a configuration example of an image-processing apparatus and a magnetic resonance imaging apparatus according to an embodiment.

FIG. 2 is an explanatory drawing of a forward propagation function of processing circuitry according to the embodiment.

FIG. 3 is an explanatory drawing of a forward propagation function of processing circuitry according to a variation of the embodiment.

FIG. 4 is a detailed diagram showing a configuration example of the magnetic resonance imaging apparatus according to the embodiment.

FIG. 5 is a flowchart showing an example of a process procedure of an image generation process in the embodiment.

FIG. 6 is a drawing showing an example of a temporary image, the number of channels as a result of a first convolution to the temporary image, and the number of channels as a result of a second convolution to the first convolution result.

FIG. 7 is a drawing showing an example of the generation of a magnetic resonance image using a result of N-th convolution by an N-th convolutional layer in the embodiment.

FIG. 8 is a drawing illustrating an example of a two-channel image which is obtained by dividing a temporary image into two, the number of channels as a result of two-channel first convolution to the two-channel image, and the number of channels as a result of two-channel second convolution to the two-channel first convolution, in a variation of the embodiment.

FIG. 9 is a drawing showing an example of the generation of a two-channel magnetic resonance image with the use of a result of two-channel N-th convolution by a N-th convolutional layer, and a magnetic resonance image obtained by synthesizing the two-channel magnetic resonance image, in a variation of the embodiment.

FIG. 10 is a diagram showing an example of a configuration of a medical signal processing apparatus according to an application example of the embodiment.

FIG. 11 is a flowchart showing an example of a procedure of the artifact reduction process in a first application example of the embodiment.

FIG. 12 is a drawing showing an example of cyclic shift processing to a magnetic resonance image with aliasing artifact along a phase encode direction, when a reduction factor is 2 in the first application example of the embodiment.

FIG. 13 is a drawing showing an example of cyclic shift processing to a magnetic resonance image with aliasing artifact along a phase encode direction, when a reduction factor is 3 in the first application example of the embodiment.

FIG. 14 is a flowchart showing an example of a procedure of an information-generation process in a second application example of the embodiment.

FIG. 15 is a drawing showing an example of an electrocardiogram waveform as a living body signal in a third application example of the embodiment.

FIG. 16 is a diagram showing an example of a medical signal processing apparatus in a fourth application example of the embodiment.

FIG. 17 is a flowchart showing an example of a procedure of an information-generation process in the fourth application example of the embodiment.

FIG. 18 is a diagram showing an example of aliasing preprocessing in the fourth application example of the embodiment.

FIG. 19 is a diagram showing an example of a medical signal processing apparatus in a fifth application example of the embodiment.

FIG. 20 is a diagram showing an example of a procedure of a coupled image generation process in the fifth application example of the embodiment.

FIG. 21 is a flowchart showing an example of aliasing post-processing in the fifth application example of the embodiment.

### DETAILED DESCRIPTION

According to one embodiment, a medical signal processing apparatus includes processing circuitry. The processing circuitry inputs a medical signal to a learned model configured to output one of the following: a corrected signal that is corrected so as to reduce a pattern of the medical signal, the pattern appearing at a location shifted by a known shift amount in a known direction; pattern-related information relating to the pattern; or disease information relating to the medical signal. The processing circuitry outputs, by using the direction and the shift amount, one of the corrected signal, the pattern-related information, or the disease information.

An object is to reduce output errors due to a learned model.

Embodiments of an image-processing apparatus and a magnetic resonance imaging apparatus will be described below in detail with reference to the drawings.

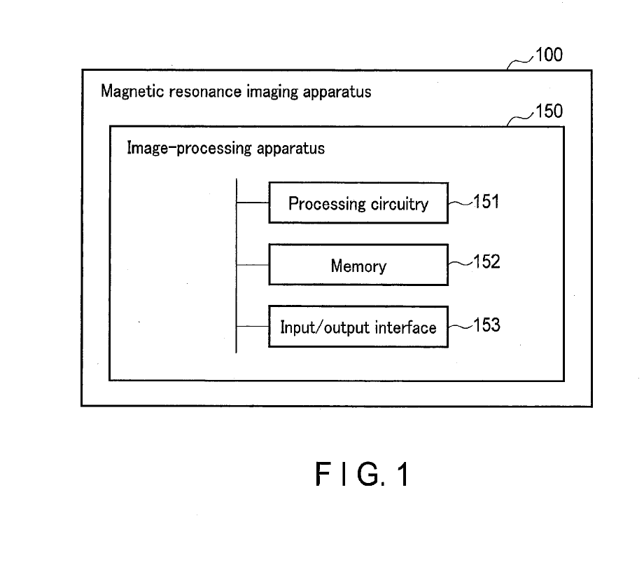

FIG. 1 is a diagram showing a configuration example of an image processing apparatus and a magnetic resonance imaging apparatus according to an embodiment. For example, as shown in FIG. 1, the magnetic resonance imaging (MRI) apparatus 100 according to the present embodiment includes an image processing apparatus 150, besides constituent elements (not shown), such as a static magnetic field magnet, gradient coils, and radio frequency coils. In the present embodiment, the image processing apparatus 150 generates a magnetic resonance image. The image-processing apparatus 150 is a device dedicated to a function of generating magnetic resonance images, or a multi-functional device. In the present embodiment, the image processing apparatus 150 will be described as a constituent element of the MRI apparatus 100, but the embodiment is not limited thereto; the functions performed by the image processing apparatus 150 may also be performed by another device communicably connected to the MRI apparatus 100. In this case, such a device serving as the image processing apparatus 150 may be installed in another location.

The image processing apparatus 150 includes processing circuitry 151, a memory 152, and an input/output interface 153. The processing circuitry 151 comprises circuitry such as a CPU (central processing unit), a GPU (graphics processing unit), an application specific integrated circuit (ASIC), a programmable logic device (for example, a simple programmable logic device (SPLD), a complex programmable logic device (CPLD), or a field programmable gate array (FPGA)), etc.

The processing circuitry 151 reads and executes a program stored in the memory 152 to realize the corresponding function. A program may be directly integrated into the processor circuitry 151, instead of stored on the memory 152. In this case, the processing circuitry 151 realizes the function by reading and executing the program integrated into the circuitry itself. The function corresponding to the program may be realized by a combination of logic circuits, not by reading and executing the program. The processing circuitry 151 of the present embodiment is not limited to a case where each processor is configured as a single circuit; a plurality of independent circuits may be combined into the processing circuitry 151 to realize its own function.

The processing circuitry 151 according to the present embodiment generates a magnetic resonance (MR) image by deep learning, which is a kind of machine learning. Deep learning in general is a kind of machine learning which makes use of a neural network with deeper layers, and which is an algorithm using nerve cells of the biological brain as a model. The processing circuitry according to the present embodiment generates an MR image using a deep learning method, particularly convolutional neural networks (CNNs). With CNNs in general, image filtering is performed in intermediate layers to target nodes that are located in neighboring regions of a pixel of interest in a previous layer, thereby extracting local characteristics of an image.

In contrast, focusing on artifact that occurs in an MR image, such as aliasing, the processing circuitry 151, according to the present embodiment, targets, for filtering, not only a node located in a neighboring region of a pixel of interest but also a node located in a region differing from the neighboring region and separated from a pixel of interest.

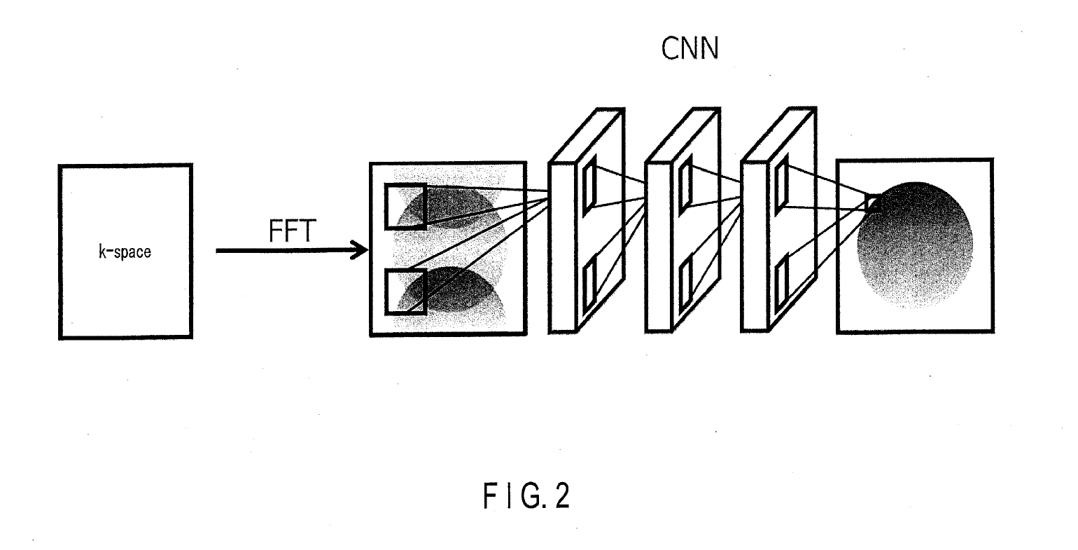

FIG. 2 is an explanatory drawing of the forward propagation function of the processing circuitry 151 according to the present embodiment. In FIG. 2, the processing circuitry 151, when applying a learned model to an MR image in which artifact has occurred, ensures that filtering is performed to target nodes located in a plurality of regions (for example, a neighboring region and a separate region as described above). Locations of these regions are determined in accordance with an imaging condition. An imaging condition includes, for example, a reduction factor indicating a degree of reduction of lines in k-space in parallel imaging (number of reduced steps), FOV (field of view), imaging parameters of a pulse sequence of the EPI method, and so on. The processing circuitry 151 calculates, for example, aliasing, or a shift due to chemical shift in the MR image in accordance with such an imaging condition, and derives a plurality of regions where the same pixel may exist within the MR image. One or more pixels are included in a region. The processing circuitry 151 performs filtering to a target node located in the derived regions. In other words, the processing circuitry 151 performs processing to each of a plurality of intermediate layers of a convolutional neural network corresponding to a learned model, so that an output from any one of the first nodes in a previous intermediate layer (which is connected to the input side of each of the intermediate layers) and an output from a second node (which is determined in the previous intermediate layer based on an imaging condition) may be input together. An MR image that is output from the learned model is an artifact-reduced image in which artifact is reduced.

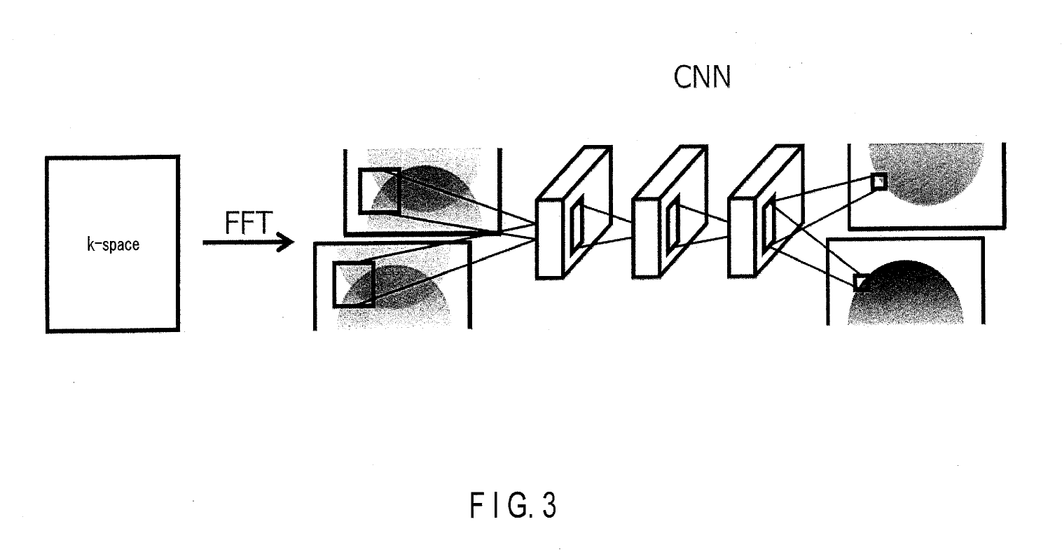

When the processing circuitry 151 derives a target for filtering in such a manner that both of a pixel in a neighboring region of a pixel of interest and a pixel in a separate region are included as the target, the derivation may be designed to be in a spatial direction as shown in FIG. 2, or in a channel direction as shown in FIG. 3. FIG. 3 is an explanatory drawing of the forward propagation function of the processing circuitry according to a variation of the embodiment.

An example of the flow of the processing performed by the MRI apparatus 100 according to the present embodiment will be described. First, the MRI apparatus 100 performs a pulse in accordance with a predetermined imaging condition, thereby collecting magnetic resonance signals and obtaining k-space data. The image processing apparatus 150 performs Fourier transform or inverse Fourier transform on the obtained k-space data, and generates an MR image. Next, the image processing apparatus 150 reads a learned model stored in the memory 152, and performs forward propagation processing to the generated MR image, thus outputting an MR image with improved image quality compared to the input image to the output interface, for example a display device, etc. The learned model used by the image processing apparatus 150 in this forward propagation processing is a model in which a location of a region targeted for filtering is specified in accordance with an imaging condition for the input image collection. For example, the image processing apparatus 150 has the memory 152 store a plurality of learned models corresponding to imaging conditions, and when forward propagation is performed, selects a learned model that matches an imaging condition from the plurality of learned models.

Hereinafter, an embodiment of the MRI apparatus and the image processing apparatus will be described in detail with reference to the drawings. In the following description, structural elements having substantially the same functions and configurations will be denoted by the same reference symbols, and repeat descriptions of such elements will be given only where necessary.

### Embodiment

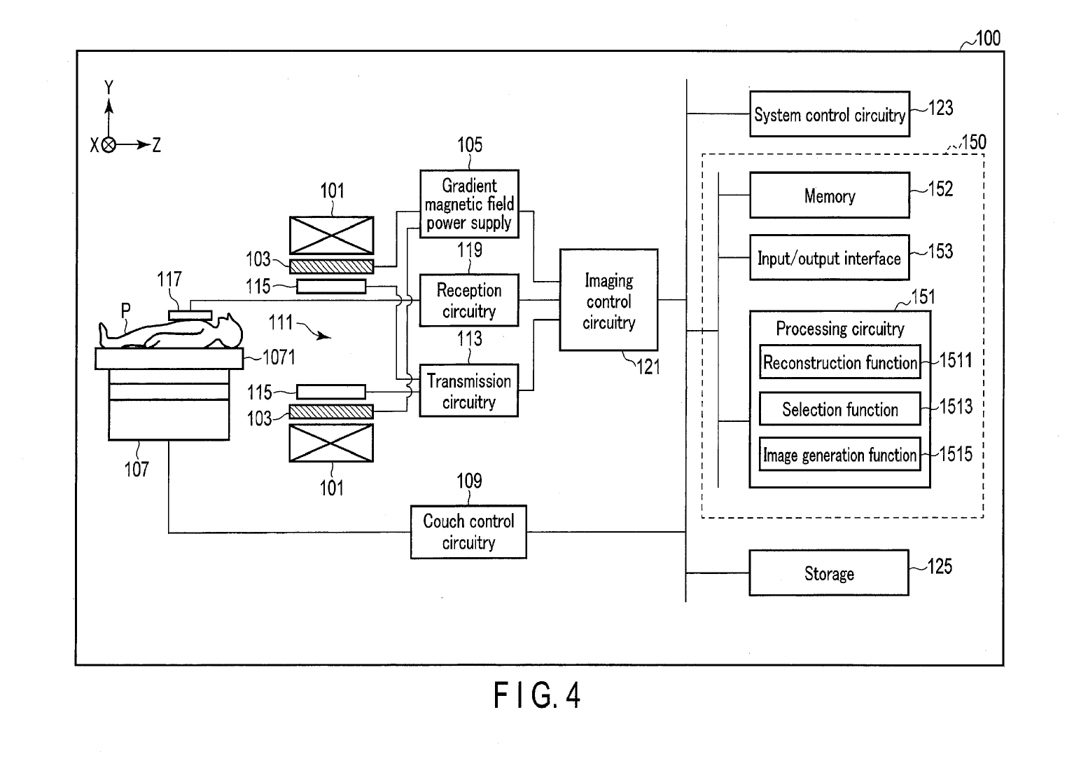

The general configuration of the MRI apparatus 100 in the present embodiment will be described with reference to FIGS. 1 and 4. FIG. 4 is a detailed diagram showing a configuration example of the MRI apparatus 100 according to the present embodiment. As shown in FIG. 4, the MRI apparatus 100 includes a static magnetic field magnet 101, a gradient coil 103, a gradient magnetic field power supply 105, a couch 107, couch control circuitry (system controller) 109, transmission circuitry (transmitter) 113, a transmitter coil 115, a receiver coil 117, reception circuitry (receiver) 119, imaging control circuitry (collector) 121, system controlling circuitry (system controller) 123, a storage device 125, and an image processing apparatus 150. A subject P is not included in the MRI apparatus 100.

The static magnetic field magnet 101 is a magnet formed in a hollow, approximately cylindrical shape. The static magnetic field magnet 101 generates an approximately uniform static magnetic field in the inner space. For example, a superconducting magnet or the like is used as the static magnetic field magnet 101.

The gradient coil 103 is a coil formed in a hollow, approximately cylindrical shape. The gradient coil 103 is arranged inside the static magnetic field magnet 101. The gradient coil 103 is formed by combining three coils respectively corresponding to the X-, Y-, and Z-axes which are orthogonal to each other. The Z-axis direction is defined as the same as the orientation of the static magnetic field. In addition, the Y-axis direction is a vertical direction, and the X-axis direction is a direction perpendicular to each of the Z-axis and the Y-axis. The three coils of the gradient coil 103 individually receive an electric current from the gradient magnetic field power supply 105 and respectively generate gradient magnetic fields in which magnetic field intensity changes along each of the X-, Y-, and Z- axes.

The gradient magnetic fields of the X, Y, and Z-axes generated by the gradient coil 103 form, for example, a gradient magnetic field for slice selection, a gradient magnetic field for phase encoding, and a gradient magnetic field for frequency encoding (also referred to as a readout gradient magnetic field). The gradient magnetic field for slice selection is used to desirably determine an imaging slice. The gradient magnetic field for phase encoding is used to change the phase of MR signals in accordance with spatial positions. The gradient magnetic field for frequency encoding is used to change the frequency of MR signals in accordance with spatial positions. The gradient magnetic fields of the X, Y, and Z-axes generated by the gradient coil 103 are used as a re-convergence pulse in which the direction of the gradient magnetic field is twice reversed in order to re-converge the phase of spins on the X-Y plane in a gradient echo method. In addition, the gradient magnetic fields of the X-, Y-, and Z-axes generated by the gradient coil 103 are used as an offset for first-order shimming of a static magnetic field.

The gradient magnetic field power supply 105 is a power supply device that supplies an electric current to the gradient coil 103 under the control of the imaging control circuitry 121.

The couch 107 is an apparatus having a couch top 1071 on which a subject P is laid. The couch 107 inserts the couch top 1071, on which the subject P is laid, into the bore 111 under the control of the couch control circuitry 109. The couch 107 is installed in, for example, an examination room in such a manner that the longitudinal axis of the couch 107 is parallel to the central axis of the static field magnet 101.

The couch control circuitry 109 is circuitry that controls the couch 107. The couch control circuitry 109 drives the couch 107 in accordance with an operator's instruction, via the input/output interface 153, to move the couch top 1071 in a longitudinal direction and a vertical direction.

The transmitter 113 supplies a high-frequency pulse modulated by a Larmor frequency to the transmitter coil 115 through the control of the imaging control circuitry 121.

The transmitter coil 115 is an RF coil provided inside the gradient coil 103. The transmitter coil 115 generates an RF pulse corresponding to a radio frequency magnetic field in accordance with an output from the transmission circuitry 113. The transmitter coil 115 is, for example, a whole-body coil (WB coil) including a plurality of coil elements. The WB coil may be used as a transmitter/receiver coil. The transmitter coil 115 may also be a WB coil made of a single coil.

The receiver coil 117 is an RF coil provided inside the gradient coil 103. The receiver coil 117 receives MR signals emitted from the subject P, caused by the high-frequency magnetic field. The receiver coil 117 outputs the received MR signals to the receiver 119. The receiver coil 117 is a coil array including, for example, one or more, typically, a plurality of coil elements. In FIG. 1, the transmitter coil 115 and the receiver coil 117 are illustrated as separate RF coils; however, the transmitter coil 115 and the receiver coil 117 may be realized by an integrated transmitter/receiver coil. The transmitter/receiver coil is, for example, a local transmitter/receiver RF coil, such as a head coil, to serve a body part targeted for imaging in the subject P.

The reception circuitry 119 generates a digital MR signal (hereinafter referred to as “MR data”) based on the MR signal that is output from the receiver coil 117 under the control of the imaging control circuitry 121. Specifically, the reception circuitry 119 performs various types of signal processing on the MR signal output from the receiver coil 117, and then performs analog-to-digital (A/D) conversion of data subjected to the various types of signal processing. The reception circuitry 119 samples the A/D-converted data, thereby generating MR data, and outputs the generated MR data to the imaging control circuitry 121.

The imaging control circuitry 121 controls, for example, the gradient magnetic field power supply 105, the transmission circuitry 113, and the reception circuitry 119 in accordance with an imaging protocol output from the processing circuitry 151 to perform imaging on the subject P. An imaging protocol includes various pulse sequences corresponding to examination. The imaging protocol defines the magnitude of the current supplied from the gradient magnetic field power supply 105 to the gradient coil 103; the timing of current supply from the gradient magnetic field power supply 105 to the gradient coil 103; the magnitude and time width of the high-frequency pulse supplied from the transmission circuitry 113 to the transmitter coil 115; the timing of high-frequency pulse supply from the transmission circuitry 113 to the transmitter coil 115; and the timing of reception of the MR signal at the receiver coil 117, etc.

The system control circuitry 123 includes, as hardware resources, a processor and a memory such as a read-only memory (ROM) and a random access memory (RAM) (both unillustrated) and controls the MRI apparatus 100 through its system control function. Specifically, the system control circuitry 123 reads a system control program stored in the storage apparatus 125, loads the program into a memory, and controls the respective circuits of the MRI apparatus 100 in accordance with the program. For example, the system control circuitry 123 reads an imaging protocol from the storage apparatus 125 based on an imaging condition that has been input by an operator through the input/output interface 153. The system control circuitry 123 may generate the imaging protocol based on the imaging condition. The system control circuitry 123 transmits the imaging protocol to the imaging control circuitry 121, and controls imaging performed to the subject P. If the image processing apparatus 150 is installed on the MRI apparatus 100, the system control circuitry 123 may be integrated into the processing circuitry 151. At this time, the system control function is performed by the processing circuitry 151, and the processing circuitry 151 functions as an alternative of the system control circuitry 123.

The storage device 125 stores various kinds of programs executed in the system control circuitry 123, various kinds of imaging protocols, imaging conditions including a plurality of imaging parameters that define imaging protocols, and the like. For example, the storage device 125 is, a semiconductor memory element, such as a RAM and a flash memory, a hard disk drive, a solid state drive, or an optical disk, etc. The storage device 125 may be a drive, etc. configured to read and write various kinds of information on a portable storage medium such as a CD-ROM drive, a DVD drive, or a flash memory, etc. If the image processing apparatus 150 is installed on the MRI apparatus 100, the data stored in the storage device 125 may be stored in the memory 152. At this time, the memory 152 functions as an alternative of the storage device 125.

The image processing apparatus 150 includes the processing circuitry 151, the memory 152, and the input/output interface 153. The processing circuitry 151 includes a reconstruction function 1511, a data arrangement function 1513, and an image generation function 1515. The various types of functions performed by the reconstruction function 1511, the selection function 1513, and the image generation function 1513 are stored in the memory 152 in a form of a computer-executable program. The processing circuitry 151 is a processor which reads a program corresponding to each of those functions from the memory 152 and executes the program to realize the function corresponding to the program. In other words, the processing circuitry 151, in a state where each of the programs is read, has a plurality of the functions etc. shown in the processing circuitry 151 of FIG. 4. The reconstruction function 1511, the selection function 1513, and the image generation function 1515 will be described later in detail.

FIG. 1 illustrates the case where the various functions are realized in a single processing circuitry 151; however, the processing circuitry 151 may be constituted by a combination of a plurality of independent processors, and the functions may be realized by the processors executing the programs. In other words, each of the above-mentioned functions may be configured as a program, and executed by a single processing circuit; alternatively, a specific function may be implemented in a dedicated independent program-execution circuit. The reconstruction function 1511, the selection function 1513, and the image generation function 1515 of the processing circuitry 151 are examples of a reconstruction unit, a selection unit, and an image generation unit, respectively. The term “processor” used in the foregoing description means, circuitry, for example, a CPU, a GPU, an ASIC, or a programmable logic device (an SPLD, a CPLD, and an FPGA). Similarly, each of the couch control circuitry 109, the transmission circuitry 113, the reception circuitry 119, and the imaging control circuitry 121, and the system control circuitry 123, etc. is also constituted by an electronic circuit such as the above-described processor.

The processing circuitry 151 fills MR data along a readout direction of k-space in accordance with an intensity of the readout gradient magnetic field by the reconstruction function 1511. The processing circuitry 151 generates an MR image by performing a Fourier transform or an inverse Fourier transform to the MR data filled in k-space. The processing circuitry 151 outputs the MR image to the memory 152 or the input/output interface 153.

The memory 152 stores MR data filled in k-space by the reconstruction function 1511, and image data etc. generated by the image generation function 1515. The memory 152 stores programs corresponding to various functions performed by the processing circuitry 151. The memory 152 is, for example, a semiconductor memory element.

The input/output interface 153 has an input interface and an output interface. The input interface has a circuit relating to, for example, a pointing device such as a mouse, or an input device such as a keyboard, an input terminal to which data is input from a network, and so on. The circuit of the input interface is not limited to a circuit relating to a physical operational component, such as a mouse or a keyboard. For example, the input interface may include an electrical signal processing circuit which receives an electrical signal corresponding to an input operation from an external input device, provided separately from the present MRI apparatus 100, and outputs the received electrical signal to various circuits. The output interface is, for example, a display, an output terminal to a network. The display displays, under the control of the system control function, various kinds of MR images reconstructed by the reconfiguration function 1511, various kinds of MR images generated by the image generation function 1515, and information related to imaging and image processing. The display is, for example, a CRT display, a liquid crystal display, an organic EL display, an LED display, a plasma display, a monitor, or any other display known in this technical field.

The overall configuration of the MRI apparatus 100 according to the present embodiment has been described above. In the following, an image generation process realized by the reconstruction function 1511, the selection function 1513, and the image generation function 1515 in the present embodiment will be described. The image generation process in the present embodiment is to apply, to the input MR image, a learned model corresponding to an imaging condition at the time when an MR image input to the image processing apparatus 150 is collected by MR imaging, to perform forward propagation to improve image quality, and to output an MR image with improved image quality.

The memory 152 stores a plurality of models, which are learned by a model learning device (not shown), as programs respectively associated with imaging conditions. The imaging conditions include the following as aforementioned: a reduction factor indicating a rate of reduction of lines in a collection of lines at equal intervals in k-space, FOV, imaging parameters of a pulse sequence of an EPI method, and the like. In the following, the learned models will be described, prior to the description of the image generation process.

A learned model is generated by an unillustrated model learning apparatus. Specifically, the model learning apparatus generates a learned model by causing a model, which has not yet been learned, to perform machine learning in accordance with a model learning program, based on learning data stored in an unillustrated learning data storage device. The model learning apparatus is a computer of a work station, etc. having a processor, such as a CPU and a GPU, etc. The model learning device and the learning data storage device may be communicably connected through a cable or a communication network, or the learning data storing device may be installed on the model learning device. In this case, learning data is supplied from the learning data storage device to the model learning device, through the cable or the communication network, etc. The model learning device and the learning data storage device may not be communicably connected. In this case, learning data is supplied from the learning data storage device to the model learning device, through a portable storage medium storing learning data thereon.

The learned model according to the present embodiment sees an MR image, in which a location where artifact occurs is known by an imaging condition, input to a CNN which then outputs the MR image from which the artifact is removed. In this case, a total number of filters in the CNN is preset. The learning data comprises: data of an MR image in which a location where artifact occurs is known by an imaging condition; data indicating a neighboring region and a separate region which are targets for filtering in the CNN, i.e., convolution processing; and data of an MR image from which artifact is removed.

The neighboring region and the separate region are preset based on a location where artifact occurs in accordance with the imaging condition. For example, if artifact is aliasing in correspondence with a reduction factor, the neighboring region and the separate region are regions which have, at their center, a location where pixels overlap due to aliasing. If artifact is N-half artifact in an EPI method, the neighboring region and the separate region are regions which have, at their center, a location where wrapped-around pixels overlap in the N-half artifact. If the artifact is chemical shift artifact, the neighboring region and the separate region are regions which have, at their center, a location where pixels shifted due to the chemical shift overlap.

The neighboring region and the separate region correspond to a plurality of convolution locations in a CNN in accordance with locations where artifact occurs due to the same physical location. For this reason, the model learning apparatus can determine a convolutional layer in a CNN in such a manner that both a neighboring region of a pixel of interest, and a separate region including a distant pixel to be superimposed on the pixel of interest, are included in the convolutional layer.

The learned model generated by the model learning apparatus is a program that causes the processing circuitry 151 to execute the following:

a neural network having a convolutional layer in which a convolution location is changed in accordance with a reduction rate at equal intervals in the reduction collection in Cartesian; a neural network having a convolutional layer in which a convolution location is changed in accordance with a location where N-half artifact occurs in echo planar imaging; a neural network having a convolutional layer in which a convolution location is changed in accordance with chemical shift; and a neural network having a convolutional layer in which a convolution location is changed in accordance with a reduction rate in the reduction collection, a location where N-half artifact occurs in echo planar imaging, and chemical shift. These neural networks are stored as programs in the memory 152 in association with corresponding imaging conditions.

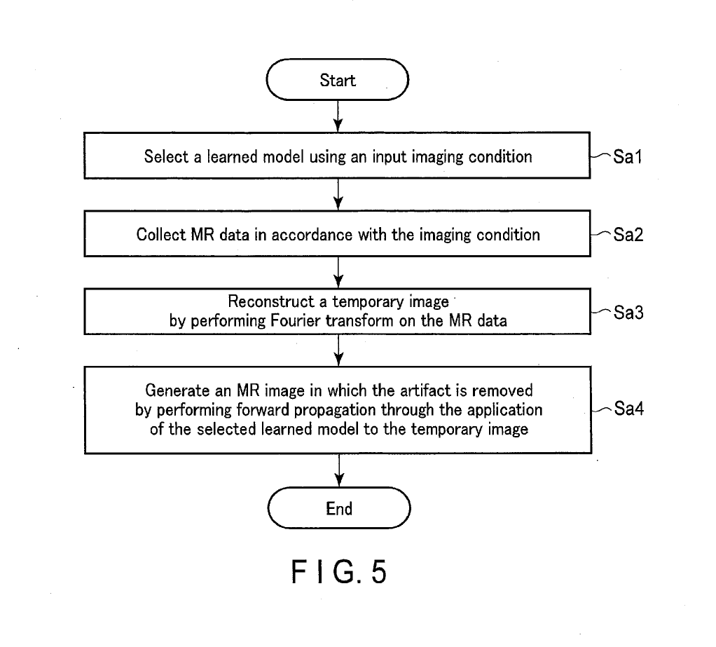

In the following, a procedure of an image generation process using a learned model will be described. FIG. 5 is a flowchart showing an example of the procedure of the image generation process. The artifact relating to the description in the present flowchart will be described on the assumption that the artifact is aliasing artifact corresponding to a reduction factor. Artifact that is adoptable in the present embodiment is not limited to aliasing artifact; for example, N-half artifact in an EPI method, or artifact due to chemical shift, is possible.

(Image Generation Process)

(Step Sa1)

An imaging condition is input in accordance with an operator's instruction via the input/output interface 153. To make the description specific, let us suppose that the imaging condition is that a reduction factor is 2. The processing circuitry 151 selects, by the selection function 1513, a learned model corresponding to the input imaging condition. The selection of a learned model may be performed after step Sa3, which will be described later.

(Step Sa2)

The imaging control circuitry 121 collects MR data by performing a pulse sequence in accordance with the input imaging condition. The imaging control circuitry 121 outputs the collected MR data to the processing circuitry 151. The processing circuitry 151 arranges the MR data in the data space, which indicates k-space, in the memory 152.

(Step Sa3)

The processing circuitry 151 reconstructs, through the reconstruction function 1511, a temporary image by performing Fourier transform or an inverted-Fourier transform on the MR data arranged in the data space, namely k-space data. Artifact occurs in the temporary image in accordance with the imaging condition. For example, if a pulse sequence is performed on the imaging condition that a reduction factor is 2, the temporary image becomes an image in which aliasing occurs in a location at a half of FOV in the phase encode direction.

(Step Sa4)

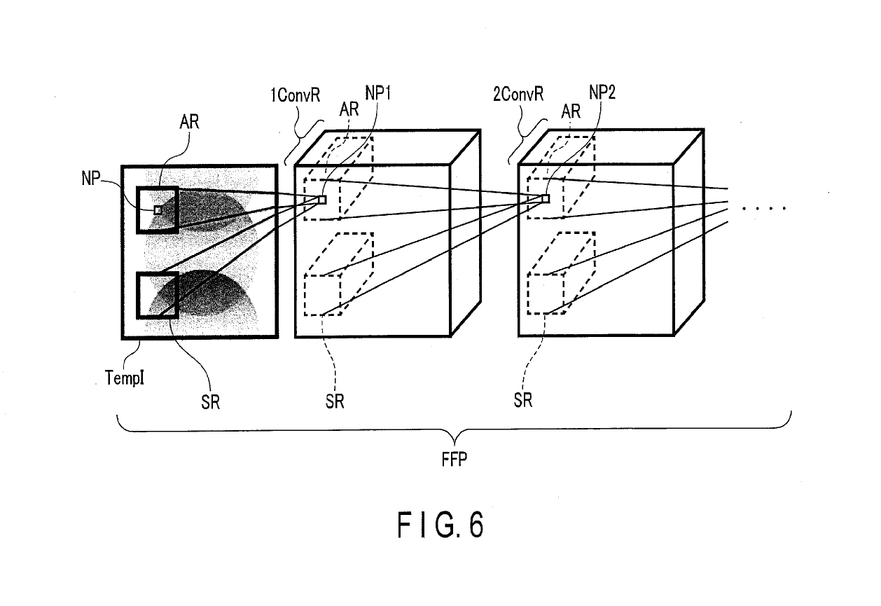

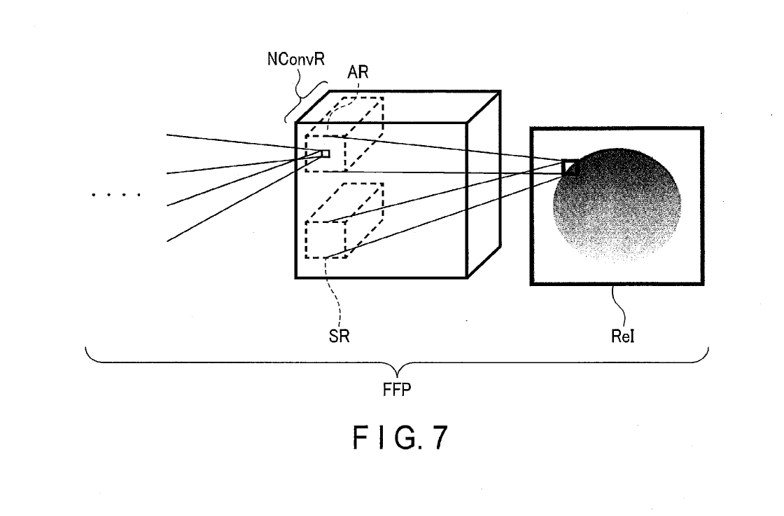

The processing circuitry 151 reads, courtesy of the image generation function 1515, a program corresponding to the selected learned model from the memory 152. The processing circuitry 151 executes a program corresponding to the read learned model. Specifically, the processing circuitry 151 applies the read learned model to the temporary image and performs forward propagation processing. The processing circuitry 151 generates an MR image from which artifact is removed as a result of the forward propagation processing. If a reduction factor is input as an imaging condition in step Sa1, the MR image generated in this step is an image from which aliasing has been removed. In the following, the forward propagation function, which performs the forward propagation processing in this step, will be described with reference to FIGS. 6 and 7.

FIGS. 6 and 7 are drawings illustrating the forward propagation shown in FIG. 2 in detail. FIG. 6 is a drawing showing an example of the temporary image TempI, the number of channels 1ConvR as a result of first convolution to the temporary image TempI, and the number of channels 2ConvR as a result of second convolution to the first convolution. To simplify the description, a pixel of interest NP in the temporary image TempI in FIG. 6 will be focused hereinafter. The pixel value of the pixel of interest NP is a sum of a pixel value due to aliasing, and an original pixel value which is not due to aliasing. For this reason, in each of the convolutional layers in the learned model, the convolution location relating to the pixel of interest NP is a neighboring region AR that includes the pixel of interest NP, and a separate region SR which has, at its center, a location of a pixel that is wrapped-around the pixel of interest NP.

In the first convolutional layer in the learned model, the processing circuitry 151 performs filtering, which corresponds to a convolution operation, via the image generation function 1515, to a plurality of pixel values included in the neighboring region AR and the separate region SR in the temporary image TempI. Specifically, the processing circuitry 151 performs a convolution operation on a plurality of pixel values included in the neighboring region AR and the separate region SR by using, as weight coefficients, a plurality of filter coefficients in each of the filters used for the first convolutional layer in the selected learned model. In the first convolution result, the processing circuitry 151 associates a product-sum value, which is a result of the convolution operation, with a location NP1 corresponding to the pixel of interest NP. The processing circuitry 151 performs the filtering processing in the first convolutional layer, in parallel, over the total number of filters in the learned model. Through the foregoing processing, the processing circuitry 151 calculates a first convolution result. For example, if the total number of filters is 64, the number of channels 1ConvR as a result of the first convolution corresponds to 64 maps.

The processing circuitry 151 inputs, via the image generation function 1515, the first convolution result to the second convolutional layer in the selected learned model. Specifically, the processing circuitry 151 performs a convolution operation to a plurality of product-sum values included in the neighboring region AR and the separated region SR in the plurality of maps corresponding to the number of channels 1ConvR as a result of the first convolution (namely the total number of channels) by using, as weight coefficients, a plurality of filter coefficients in each of the filters used in the second convolutional layer. For the convolution coefficients of the neighboring region AR and the separate region SR, these may be learned through use of different coefficients, or indeed the same coefficients. The convolution range (kernel size) is not necessarily of a square shape; for example, it may be a shape in accordance with an aspect ratio of the image, or a shape elongated in a readout direction. In the second convolution result, the processing circuitry 151 associates the product-sum values, which are a result of the convolution calculation by the second convolutional layer, with the location NP2 corresponding to the pixel of interest NP. The processing circuitry 151 performs the filtering processing in the second convolutional layer, in parallel, over the total number of filters in the learned model. Through the foregoing processing, the processing circuitry 151 calculates a second convolution result. Hereinafter, similar to the above, the processing circuitry 151 repeats the calculation through the filtering in the forward propagation processing FFP, over the total number N of the convolutional layers in the learned model. A pooling layer, an activation layer, a contrast normalization layer, a shortcut (ResNet), a coupling with previous data (DenseNet), and the like may be provided between two adjacent convolutional layers, as appropriate.

FIG. 7 is a drawing showing an example of the generation of an MR image ReT using an N-th convolution result by an N-th convolutional layer. As shown in FIG. 7, the processing circuitry 151 generates, courtesy of the image generation function 1515, an MR image ReI by adopting a fully-connected layer to the number of channels NConvR which correspond to a plurality of N-th convolution results. The processing circuitry 151 outputs the generated MR image ReI to the input/output interface 153. The display of the input/output interface 153 displays the generated MR image ReI.

According to the above-described configuration, the following advantageous effects can be obtained.

According to the MRI apparatus 100 in the present embodiment, it is possible to output an MR image with improved image quality through the application of a learned model corresponding to an imaging condition, adopted at the time of collecting an input MR image by MR imaging to said input MR image, and performance of forward propagation for improving image quality. Specifically, according to the MRI apparatus 100, it is possible to generate an MR image through the following operations: collecting MR data through reduction collection at equal intervals in k-space; reconstructing a temporary image by Fourier transform on the MR data, selecting, based on an imaging condition relating to the temporary image, a learned model to be applied to the temporary image, from a plurality of learned models having convolutional layers learned through the use of a plurality of convolution locations, set in accordance with a location where artifact occurs due to a same physical location, and respectively corresponding to imaging conditions; and applying the selected learned model to the temporary image.

According to the MRI apparatus 100, it is possible to generate an MR image in which artifact that occurs at a known location depending on an imaging condition is removed, with the use of at least one of the following elements making up the plurality of learned models: a neural network having a convolutional layer in which a convolution location is changed in accordance with a reduction rate in reduction collection; a neural network having a convolutional layer in which a convolution location is changed in accordance with a location where N-half artifact occurs in echo planar imaging; or a neural network having a convolutional layer in which a convolution location is changed in accordance with chemical shift.

Thus, according to the MRI apparatus 100, a convolutional layer is designed through learning of non-linear mapping with the use of, as information necessary in terms of effectiveness and efficiency, a convolution location corresponding to a known aliasing location in accordance with an imaging condition; in other words, a neighboring region AR and a separate region SR, and a learned model having the designed convolutional layer, thereby generating an MR image in which artifact is removed. It is thus possible to improve image quality of a reconstructed MR image according to the present MRI apparatus 100.

(Variation of Present Embodiment)

A difference between the present variation and the foregoing embodiment lies in that a temporary image divided into two (hereinafter, “two-channel image”) in accordance with a location where artifact occurs (i.e., a location of aliasing) is used as a two-channel input into a CNN to generate an MR image in which artifact is removed. The input into a CNN is not limited to such a two-channel image, and may be a multiple-channel image divided into multiple parts in accordance with a location where artifact occurs. First, a learned model in the present variation will be described, and the generation of an MR image using a learned model in the present variation will then be described.

The learned model in the present variation is a CNN to which a two-channel image is input, and is obtained by dividing a temporary image in which a location of artifact is known by an imaging condition, and which outputs an MR image in which artifact is removed in the MR image. The learning data is data obtained by dividing an MR image for which a location where artifact occurs is known by an imaging condition, data indicating a neighboring region and a separate region, and data of an MR image from which artifact is removed. The image input into a CNN is doubled compared to the foregoing embodiment, from one to two channels. The model learning apparatus generates a learned model pertaining to the present variation through learning of a CNN with the use of learned data. The generated learned model is stored in the memory 152 as a program, along with a corresponding imaging condition.

The processing circuitry 151 divides, through the image generation function 1515, the temporary image into two in step Sa3. The processing circuitry 151 generates a two-channel image by dividing the temporary image into two along an axis in which no aliasing occurs in the temporary image. The axis dividing the temporary image into two can be discretionarily set, and is not limited to the above-mentioned axis. The processing circuitry 151 generates an MR image from which artifact is removed by performing the forward propagation processing through the application of the selected learned model to the two-channel image in step Sa4. To make the description specific, suppose artifact in the description of the present variation hereafter is aliasing corresponding to a reduction factor. Artifact that is adoptable in the present variation is not limited to aliasing artifact; for example, N-half artifact in an EPI method, or artifact due to chemical shift is possible.

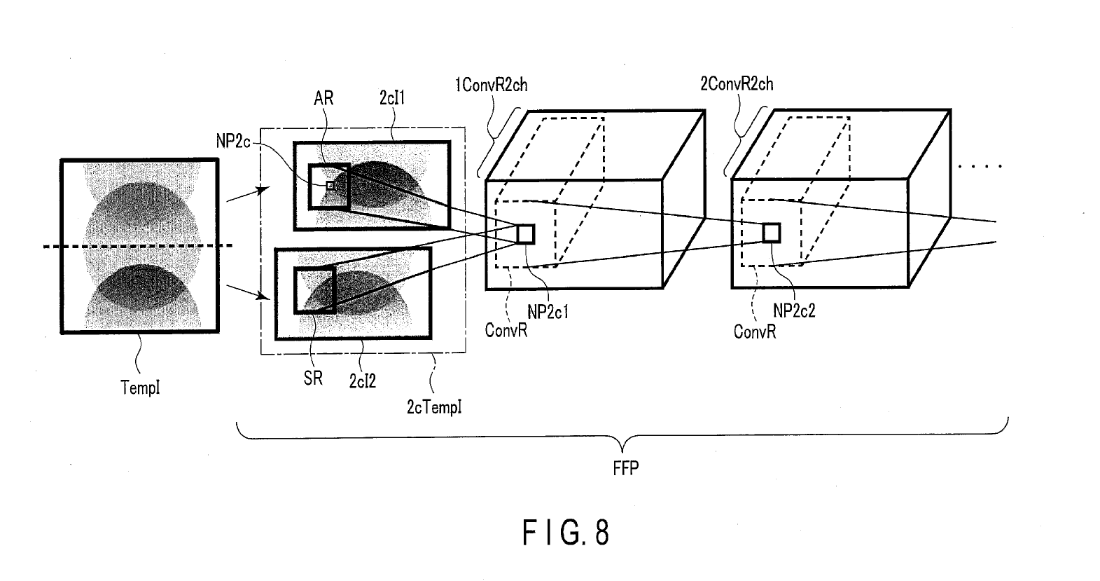

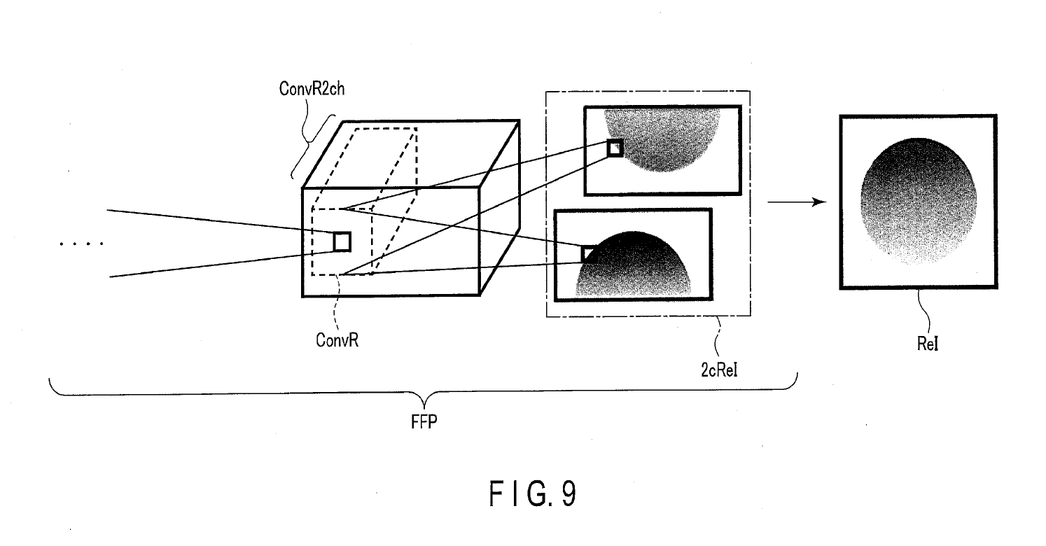

FIGS. 8 and 9 are drawings illustrating the forward propagation shown in FIG. 3 in detail. FIG. 8 is a drawing illustrating an example of a two-channel image 2cTempI which is obtained by dividing a temporary image TempI into two, the number of channels 1ConvR2ch as a result of a two-channel first convolution to the two-channel image 2cTempI, and the number of channels 2ConvRch2 as a result of a two-channel second convolution to the two-channel first convolution. To simplify the description, a pixel of interest NP2c in the two-channel image 2cTempI in FIG. 8 will be focused on hereinafter. The pixel value of the pixel of interest NP2c is a sum of a pixel value due to aliasing and an original pixel value which is not due to aliasing. For this reason, the convolution location relating to the pixel of interest NP2c in the first image 2cI1 of the two-channel image 2cTempI is a neighboring region AR, in which the pixel of interest NP2 is centered in the first image 2cI1, and a separate region SR in which a pixel location wrapped-around the pixel of interest NP2 is centered in the second image 2cI2 of said two-channel image 2cTempI.

In the first convolutional layer in the learned model, the processing circuitry 151 performs filtering, which corresponds to a convolution operation, through the image generation function 1515, to a plurality of pixel values included in the neighboring region AR of the first image 2cI1 and a plurality of pixel values included in the separate region SR in the second image 2cI2. Specifically, the processing circuitry 151 performs a product-sum operation, namely a convolution operation, to a plurality of pixel values included in the neighboring region AR and the separate region SR by using, as weight coefficients, a plurality of filter coefficients in each of the filters used for the first convolutional layer in the selected learned model. In the two-channel first convolution result, the processing circuitry 151 associates a product-sum value, which is a result of the convolution operation, with a location NP2c1 corresponding to the pixel of interest NP2c. The processing circuitry 151 performs the filtering processing in parallel, over the total number of filters in the learned model. Through the foregoing processing, the processing circuitry 151 calculates a two-channel first convolution result. For example, if the total number of filters is 128, the number of channels 1ConvR2ch as a result of the two-channel first convolution corresponds to 128 maps.

The processing circuitry 151 inputs, through the image generation function 1515, the first convolution result into the second convolutional layer in the selected learned model. Specifically, the processing circuitry 151 performs a convolution operation to a plurality of product-sum values by using, as weight coefficients, a plurality of filter coefficients in each of the filters used in the second convolutional layer. The plurality of product-sum values is included in the region ConvR in which the location NP2c1 in the plurality of maps corresponding to the number of channels 1ConvR2ch as a result of the first convolution, namely the total number of filters, is centered. In the two-channel second convolution result, the processing circuitry 151 associates the product-sum values, which is a result of the convolution operation by the second convolutional layer, with the location NP2c2 corresponding to the pixel of interest NP. The processing circuitry 151 performs the filtering processing in the second convolutional layer in parallel, over the total number of filters in the learned model. Through the foregoing processing, the processing circuitry 151 calculates a two-channel second convolution result. Hereinafter, similar to the above, the processing circuitry 151 repeats the calculation through filtering in the feed-forward processing FFP, over the total number N of the convolutional layers in the learned model. A pooling layer, a local contrast normalization layer, and the like may be provided between adjacent two convolutional layers, as appropriate.

FIG. 9 is a drawing showing an example of the generation of a two-channel magnetic resonance image 2cReI using a result of two-channel N-th convolution by an N-th convolutional layer, and an MR image ReI obtained by synthesizing the two-channel MR image 2cReI. As shown in FIG. 9, the processing circuitry 151 generates, through the image generation function 1515, a two-channel MR image 2cReI by adopting a fully-connected layer to the number of channels NConvRch corresponding to a plurality of N-th convolution results. The processing circuitry 151 synthesizes the generated two-channel MR image 2cReI to generate an MR image ReI in which artifact is removed.

(First Modification)

A difference between the first modification and the foregoing embodiment lies in that a complex image is used as a temporary image, and a complex operation is used as a convolution operation in a convolutional layer. In other words, a calculation in a CNN in the present modification is executed as a complex operation in a complex space. The learned model in the present modification is a CNN that executes a complex operation to an input of a complex image in which a location of artifact is known from an imaging condition, and then outputs a complex image in which artifact is removed. The learning data is data of a complex image in which a location where artifact occurs is known from an imaging condition, data indicating a neighboring region and a separate region, and data of a complex image from which artifact is removed. The model learning apparatus generates a learned model pertaining to the present variation through learning a CNN with the use of learned data. The generated learned model is stored in the memory 152 as a program, along with a corresponding imaging condition.

The processing circuitry 151 performs quadrature phase detection to a collected MR signal by the reconstruction function 1511, and generates complex MR data. The processing circuitry 151 generates a complex image by performing a Fourier transform or an inverse Fourier transform on the complex MR data. The processing circuitry 151 generates, through the image generation function 1515, a complex image from which artifact is removed, by performing the forward propagation processing through the application of the selected learned model to the complex image. The processing circuitry 151 generates an MR image by using the complex image generated by the forward propagation processing.

(Second Modification)

A difference between the second modification and the foregoing variation lies in that a real-part image and an imaginary-part image in a complex image are used as the two-channel image described in the variation. The learned model according to the present modification is a CNN which has a real-part image and an imaginary-part image in which a location where artifact occurs is known by an imaging condition, input, and then outputs a real-part image and an imaginary-part image in which artifact is respectively removed from the aforementioned. The learning data is data of a real-part image and an imaginary-part image in which a location where artifact occurs is known from an imaging condition, data indicating a neighboring region and a separate region, and data of the real-part image and the imaginary-part image in which artifact is respectively removed. The model learning apparatus generates a learned model pertaining to the present variation through learning a CNN with the use of learned data. The generated learned model is stored in the memory 152 as a program, along with a corresponding imaging condition.

The processing circuitry 151 performs quadrature phase detection to a collected MR signal by the reconstruction function 1511, and generates complex MR data. The processing circuitry 151 generates a real-part image by performing a Fourier transform or an inverse Fourier transform on the complex MR data. The processing circuitry 151 generates an imaginary-part image by performing a Fourier transform or an inverse Fourier transform on the complex MR data. The processing circuitry 151 generates, by the image generation function 1515, a real-part image and an imaginary-part image from which artifact is removed, by performing the forward propagation processing through the application of the selected learned model on the real-part image and the imaginary-part image. The processing circuitry 151 generates an. MR image by using the real-part image and imaginary-part image generated by the forward propagation processing.

As modifications of the foregoing embodiment, in the event the technical idea of the present image processing apparatus 150 is realized by cloud computing or the like, a server on the Internet may have the processing circuitry 151 and the memory 152 shown in FIG. 1 and FIG. 4, for example. In this case, the reconstruction function 1511, the selection function 1513, the image generation function 1515, etc. are realized through installation of an image processing program that executes these functions onto the processing apparatus 151 of a server and expands these functions in a memory. For example, the server can perform the image generation process, etc.

According to at least one of the foregoing embodiment, the variation, and the modifications, it is possible to improve image quality of an MR image.

### FIRST APPLICATION EXAMPLE

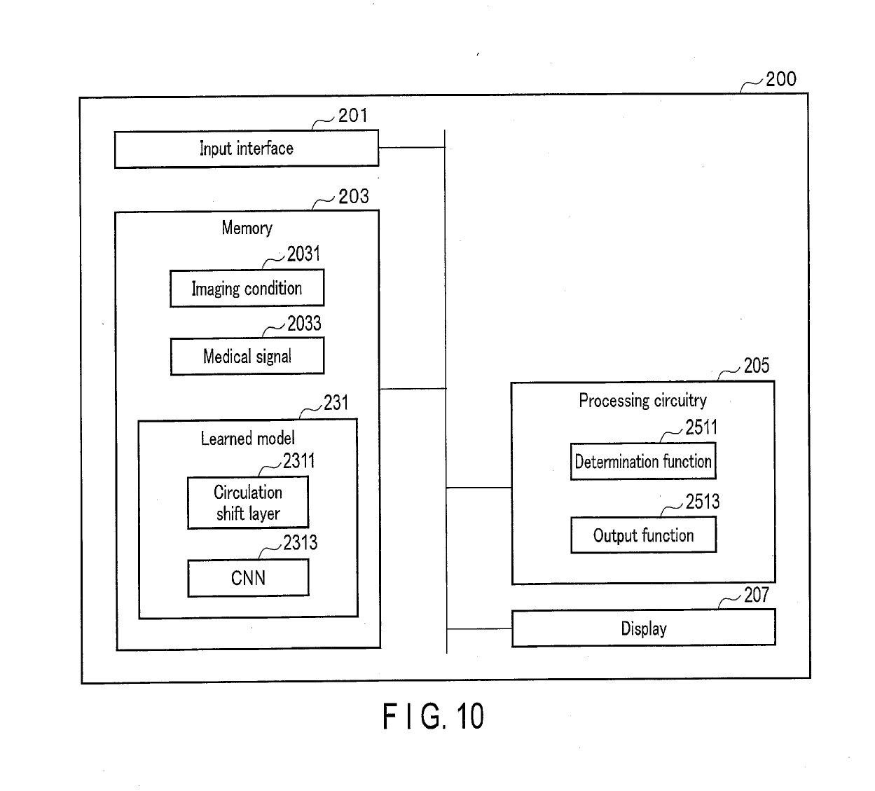

A first application example of the embodiment will be explained below. FIG. 10 is a diagram showing an example of a configuration of a medical signal processing apparatus 200 according to the present application example. As shown in FIG. 10, the medical signal processing apparatus 200 has an input interface 201, a memory 203, processing circuitry 205, and a display 207. The medical signal processing apparatus 200 may be installed on the MRI apparatus 100. Since the input interface 201 and the display 207 correspond to the input/output interface 153 of the foregoing embodiment, descriptions thereof are omitted. The input interface 201 may function as a communication interface that obtains imaging conditions and medical images from a medical image diagnostic apparatus such as an MRI apparatus. Since hardware configuration of the memory 203 and the processing circuitry 205 are the same in the present embodiment, the description thereof is omitted. The processing circuitry 205 may have an acquisition function for acquiring imaging conditions and medical images from modality (not shown). The processing circuitry 205, realizing the acquisition function, functions as an acquisition unit.

The memory 203 stores a learned model 231, imaging conditions 2031, and medical signals 2033. The learned model 231 is configured to output at least one of the following: a corrected signal obtained by correcting a medical signal 2033 having a pattern, which appears at a location shifted by a known amount in a known direction, so as to reduce said pattern; pattern-related information that relates to a pattern; or disease information relating to the medical signal 2033. The medical signal 2033 is an MR image generated by magnetic resonance imaging performed to a subject P, and corresponds to the above-described temporary image. Hereinafter, in order to make the description specific, suppose the medical signal 2033 is an MR image. The pattern is, for example, artifact that occurs in an MR image in accordance with an imaging condition 2031 of magnetic resonance imaging. Artifact is, for example, at least one of aliasing, N-half artifact, chemical shift artifact, or motion artifact.

In the description hereafter, suppose that the learned model 231 in the present application example outputs a corrected signal. The case where the learned model 231 outputs pattern-related information or disease information will be later described in the second application example. The case where the pattern is non-artifact, and the medical signal 2033 is a non-two-dimensional medical signal (for example, non-image) will be described later in the third application example. The processing pertinent to the present application example is suitable for denoising of medical signals, but may be used for a purpose different from denoising, as will be described in the second and third application examples.

The known direction is a direction relating to occurrence of artifact, and is defined by a pulse sequence in the imaging condition 2031. For example, if artifact is aliasing artifact caused by parallel imaging, the known direction corresponds to a wrap-around direction in an MR image. The wrap-around direction is not limited to a phase encode direction, and may be a direction defined by both of a phase encode direction and a frequency encode direction, as in the techniques of multi-slice CAIPIRINHA and two-dimensional CAIPIRINHA. If artifact is N-half artifact caused by the performance of pulse sequence of the EPI method, the known direction corresponds to a phase encode direction in an MR image, for example. If artifact is chemical artifact, the known direction corresponds to a frequency encode direction in an MR image, for example. If artifact is motion artifact, the known direction is, for example, a direction of a body movement of a subject P, or a direction of pulsation flow in an MR image, for example.

The known shift amount is an amount of a cyclic translation of an MR image based on a location where artifact occurs, and is defined by a pulse sequence in the imaging condition 2031. For example, if the artifact is aliasing artifact, the known shift amount corresponds to a reduction factor in a pulse sequence. The artifact is N-half artifact, the known shift amount corresponds to a location where ghost appears in a phase encode direction in an MR image, for example. If the artifact is chemical shift artifact, the known shift amount is dependent on a difference between a resonance frequency of water and a resonance frequency of fat, and an intensity of a static magnetic field, for example. If the artifact is motion artifact, the known shift amount corresponds to a location where ghost appears in a phase encode direction in an MR image, for example. The aliasing location relating to the known direction and the known shift amount is defined or estimated as a location where artifact occurs, based on the imaging condition 2031.

The corrected signal corresponds to an MR image in which artifact is reduced (hereinafter, “artifact-reduced image”). For example, if artifact as a pattern is aliasing artifact, the corrected signal corresponds to an artifact-reduced image in which aliasing artifact is reduced. The artifact-reduced image is an image corresponding to the MR image ReT shown in FIGS. 7 and 9.

The learned model 231 outputs a corrected signal from an input MR image, with the use of the known direction and the known shift amount. Specifically, the learned model 231 has a circulation shift layer 2311 and a CNN 2313 as an example of a deep neural network (DNN), as shown in FIG. 10. The learned model 231 is stored in the memory 203 in association with the imaging condition 2031. In other words, the memory 203 stores a plurality of learned models in accordance with the known direction and the known shift amount. The circulation shift layer 2311 is preset based on a known direction and a known shift amount which are related to a location where artifact occurs, in accordance with the imaging condition 2031. In other words, the circulation shift layer 2311 is a model that is not machine-learned. The CNN 2313, on the other hand, is a model generated by machine learning. The learned model 231, which is a combination of the circulation shift layer 2311 (not machine-learned) and the CNN 2313, which is generated through machine learning, is generated by machine learning.

The circulation shift layer 2311 generates a shift signal by cyclically shifting an MR image by the known shift amount along the known direction. The shift signal corresponds to an image obtained by cyclically shifting the MR image by the known shift amount along the known direction (hereinafter “shift image”). Cyclical shifting corresponds to circulation of a medical signal on the assumption that one end of the medical signal continues to the other end with respect to a known direction. The processing performed by the circulation shift layer 2311 may be realized by deep learning. In other words, the circulation shift layer 2311 may be realized by a DNN that outputs a shift image from an input of an approximately-cyclic MR image as an input. The processing performed by the circulation shift layer 2311 will be described later.

The CNN 2313 is a neural network configured to output a corrected signal with the use of an MR signal and a shift image. A neural network having a local connectivity may be used instead of the CNN 2313. For example, if data output from the learned model 231 is an artifact-reduced image, the learned model 231 has the circulation shift layer 2311 and the CNN 2313. A DNN as a full connection may be used as appropriate instead of the CNN 2313, in accordance with a purpose of data output from the learned model 231. As the CNN 2313 or the DNN in the present embodiment and application examples, ResNet (residual network), DenseNet (dense convolutional network), or U-Net may be used. In the ResNet, DenseNet, or U-Net, etc., a combination of the circulation shift layer 2311 and the CNN 2313 may be repeated as appropriate.

The processing circuitry 205 has a determination function 2511 and an output function 2513. The processing circuitry 205 determines, through the determination function 2511, a known direction and a known shift amount based on the imaging condition 2031 for a subject P. The processing circuitry 205 determines a learned model based on the determined direction and shift amount. The processing circuitry 205 inputs an MR image to the determined learned model, and outputs, through the output function 2513, a corrected signal by using the known direction and the known shift amount. The processing circuitry 205 corresponds to a processor, and is composed of an electronic circuit, such as the aforementioned processor.

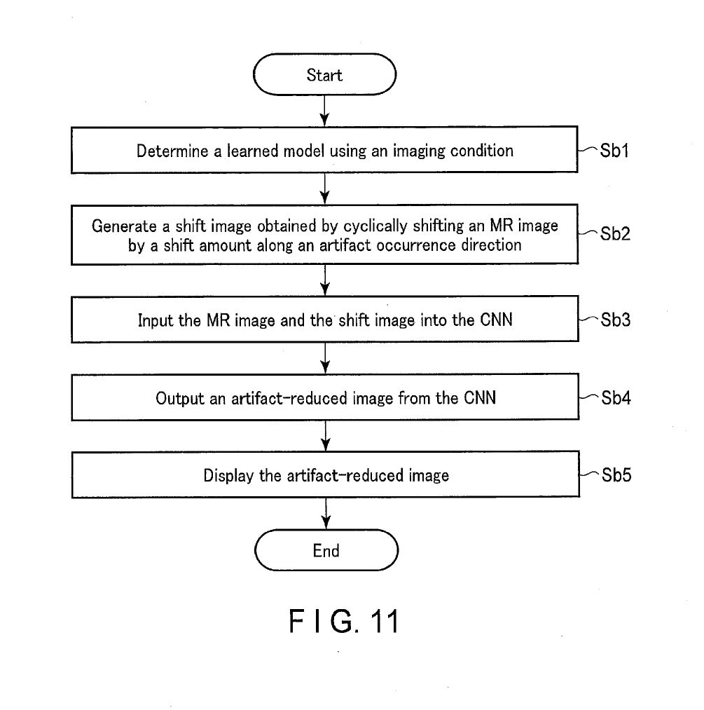

In the following, a procedure of the generation of an artifact-reduced image as a corrected signal (hereinafter, “artifact reduction process”) with the use of the learned model 231 in the present application example will be described with reference to FIGS. 11 to 13. FIG. 11 is a flowchart showing an example of the artifact reduction process.

(Artifact Reduction Process)

(Step Sbl)

The processing circuitry 205 determines, through the determination function 2511, a learned model to which an MR image is input, based on the imaging condition 2031. Specifically, the processing circuitry 205 determines a direction in which a pattern, such as artifact, appears (corresponding to a known direction, and referred to as “artifact occurrence direction” hereinafter), and a shift amount of the pattern in a known direction (hereinafter, “shift amount”), based on the imaging condition 2031 which is used for obtaining the MR image. Specifically, the processing circuitry 205 checks an imaging parameter associated with an MR image against a correspondence table of a direction corresponding to an imaging parameter of a pulse sequence and a shift amount (hereinafter, “direction-shift amount correspondence table”). The processing circuitry 205 determines an artifact occurrence direction and a shift amount through the check using the direction-shift amount correspondence table. The direction-shift amount correspondence table is stored in the memory 203 in advance, and read by the determination function 2511 from the memory 203 before being output to the processing circuitry 205.

The processing circuitry 205 checks, through the determination function 2511, the artifact occurrence direction and the shift amount against a correspondence table of learned models corresponding to directions and shift amounts (hereinafter, “model correspondence table”). The processing circuitry 205 determines a learned model through the checking with the model correspondence table. The model correspondence table is stored in the memory 203 in advance. The processing circuitry 205 reads the determined learned model from the memory 203 and outputs the same to the processing circuitry 205. The processing circuitry 205 that realizes the determination function 2511 corresponds to a determination unit.

(Step Sb2)

The processing circuitry 205 generates, through the output function 2513, a shift image via the circulation shift layer 2311 of the learned model 231. Specifically, the processing circuitry 205 inputs an MR image into the circulation shift layer 2311. The circulation shift layer 2311 generates a shift image which is obtained by cyclically shifting the input MR image by the shift amount along the artifact occurrence direction.

The process performed to an MR image by the circulation shift layer 2311 (hereinafter, “circulation shift processing”) will be described with reference to

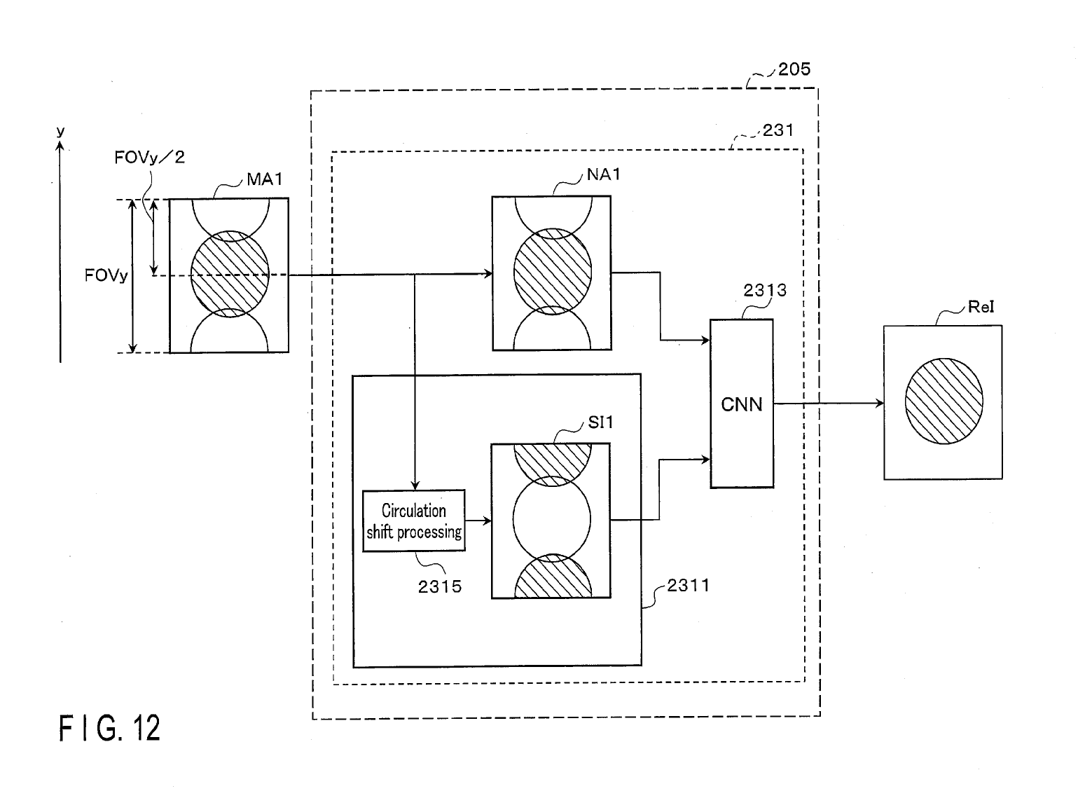

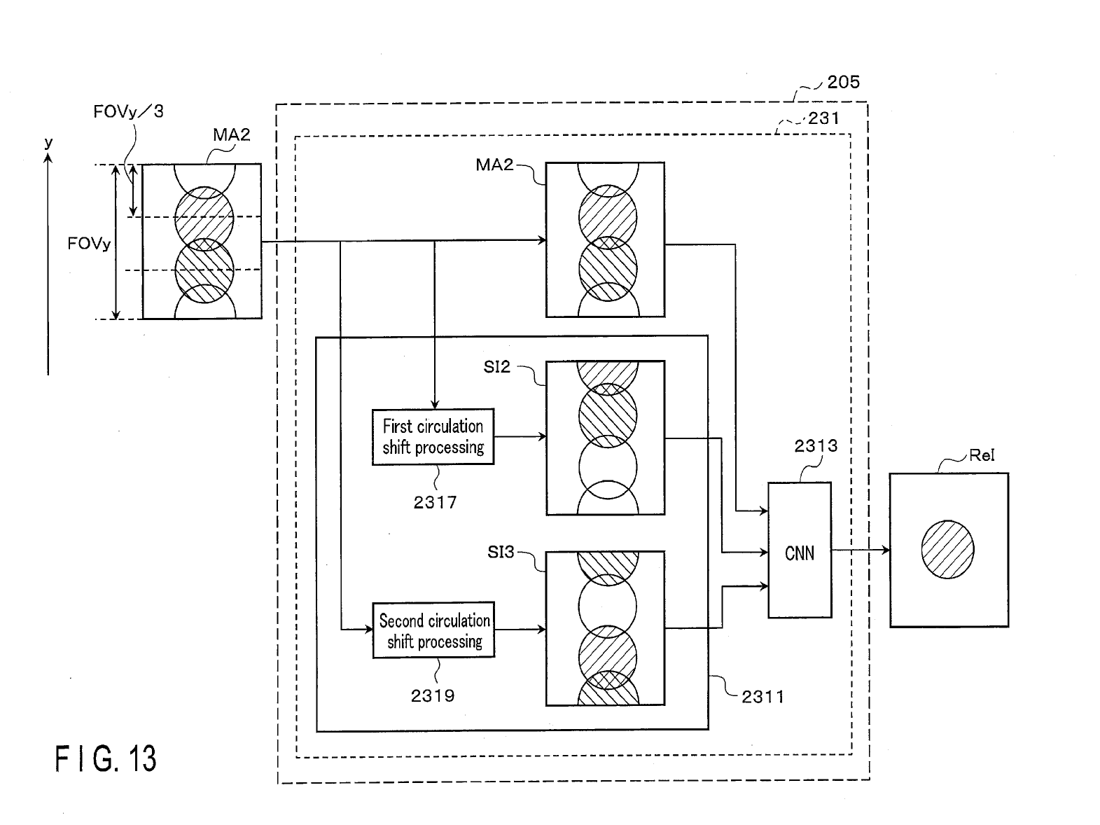

FIGS. 12 and 13. FIG. 12 is a drawing showing an example of circulation shift processing to a magnetic resonance image MA1 having aliasing artifact along a phase encode direction, when a reduction factor is 2. In the MR image MA1 shown in FIG. 12, the artifact occurrence direction is a phase encode direction (y direction), and the shift amount is half of FOVy in a vertical direction, (i.e., FOVy/2). In this case, the circulation shift processing 2315 generates a shift image SI1 by cyclically translating the MR image MA1 for FOVy/2 along the y direction.

FIG. 13 is a drawing showing an example of circulation shift processing to an MR image MA2 having aliasing artifact along a phase encode direction, when a reduction factor is 3. In the MR image MA2 shown in FIG. 13, the artifact occurrence direction is a phase encode direction (y direction), and the shift amount is one-third of FOVy in a vertical direction, (i.e., FOVy/3). In this case, the first circulation shift processing 2317 generates a shift image SI2 by cyclically translating the MR image MA2 by FOVy/3 along the y direction. In this case, the second circulation shift processing 2319 generates a second shift image SI3 by cyclically translating the MR image MA2 by FOVy/3 along the y direction. The known shift amount used for the cyclic shifting is not limited to those shown in FIGS. 12 and 13, and the shift amount may change in accordance with a type of artifact or a status of artifact.

(Step Sb3)

The processing circuitry 205 inputs the MR image to the CNN 2313, through the output function 2513, along with the shift image. For example, if the MR image MA1 is the one shown in FIG. 12, the processing circuitry 205 inputs the generated shift image SI1 to the CNN 2313, along with the MR image MA1. If the MR image MA2 is the one shown in FIG. 13, the processing circuitry 205 inputs a first shift image SI2 and a second shift image SI3 into the CNN 2313, together with the MR image MA1.

(Step Sb4)

The processing circuitry 205 outputs, through the output function 2513, an artifact-reduced image ReI as a corrected signal, from the CNN 2313 to which the shift image output from the circulation shift layer 2311 and the MR image have been input. The processing circuitry 205 outputs the artifact-reduced image ReI to the memory 203 and the display 207.

(Step Sb5)

The display 207 displays the artifact-reduced image ReI. The artifact-reduced image ReI is an MR image in which aliasing artifact is reduced, as shown in FIGS. 12 and 13. The processing circuitry 205 may output an artifact-reduced image ReI to an external device, such as a medical image storage device, via a network (not shown).

According to the above-described configuration, the following advantageous effects can be obtained.

According to the medical signal processing apparatus 200 in the present application example, a medical signal 2033 is input to a learned model, configured to output a corrected signal obtained by correcting the medical signal 2033 having a pattern which appears at a location shifted for a known shift amount along a known direction, so as to reduce said pattern; and the corrected signal is output with the use of the known direction and the known shift amount. The learned model in the present medical signal processing apparatus 200 has a circulation shift layer 2311 that generates a shift signal obtained by cyclically shifting the medical signal 2033 for a known shift amount along a known direction, and a neural network 2313 configured to output a corrected signal by using the medical signal 2033 and the shift signal.

According to the medical signal processing apparatus 200 relating to the present application example and the foregoing embodiment, the medical signal 2033 is an MR signal generated by magnetic resonance imaging performed to a subject; the pattern is artifact caused in an MR image in accordance with an imaging condition 2031 of magnetic resonance imaging; the corrected signal is an artifact-reduced image in which artifact is removed; the learned model 231 is a convolutional neural network having a plurality of intermediate layers; and the model processes, as one input, an output from a first node of any of the intermediate layers of a previous stage connected to the input side of the current intermediate layers, and an output from a second node determined by an imaging condition 2031 among the intermediate layers of the previous stage.

Further according to the medical signal processing apparatus 200, the medical signal 2033 is an MR image generated by medical resonance imaging performed to a subject P; the pattern is artifact caused in an MR image in accordance with an imaging condition 2031 of magnetic resonance imaging; the corrected signal is an artifact-reduced image ReI in which artifact is reduced; the neural network is a neural network having local linear couplings in each of intermediate layers; and the known direction is a direction relating to occurrence of artifact, and the known shift amount is an amount of translation based on a location where artifact occurs. According to the medical signal processing apparatus 200, artifact is at least one of aliasing, N-half artifact, chemical shift artifact, or motion artifact.

Thus, according to the medical signal processing apparatus 200, it is possible to generate an MR image with reduced artifact, which appears at a location shifted by a known shift amount along a known direction based on an imaging condition 2031, improve image quality of the MR image, and increase efficiency of diagnosis of a subject P.

### SECOND APPLICATION EXAMPLE

A difference between the second application example and the first application example lies in that the learned model 231 outputs pattern-related information or disease information. In the learned model 231 of the present application example, a DNN may be used instead of the CNN 2313. In the learned model 231 of the present application example, a ½ max pool layer may be added after the CNN 2313 (or the DNN) in ResNet, DenseNet, or U-Net. In this case, a full-connection DNN may be provided at the final stage of the learned model 231, so that the learned model 231 outputs pattern-related information or disease information.

If the pattern is artifact, the pattern-related information is, for example, data indicating at least one physical parameter used for correcting an MR image, or presence/absence (detection result) of artifact. For example, if magnetic resonance imaging is performed to a subject P with the use of a pulse sequence of the EPI method, the physical parameter corresponds to an estimated value of a physical quantity indicating a delay amount of occurrence of a gradient field. The data indicating presence/absence of artifact is, for example, two values (0 or 1) indicating presence/absence of respective artifact in an MR image.

The disease information is data indicating a result of identification of each disease in an MR image. For example, if an MR image has artifact appearing at a location shifted by a known shift amount along a known shift direction, the disease information corresponds to an index value indicating a degree of each disease in the MR image. In other words, the disease information has an index value indicating a level of likelihood for each disease. The index value may be a value indicating presence/absence of a disease in a medical signal. Data indicating presence/absence of an index value and artifact is output as a percentage, through incorporation of a sigmoid function into the learned model 231.

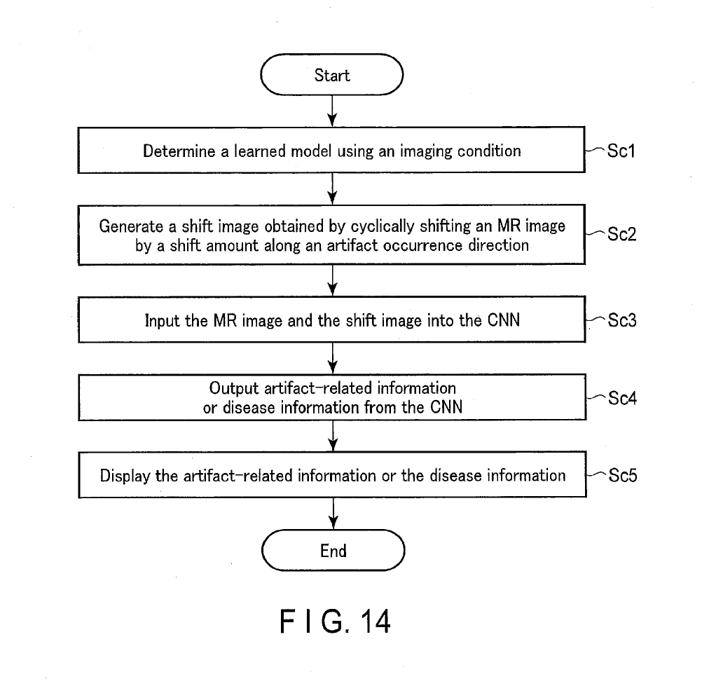

In the following, a procedure of the generation of pattern-related information or disease information with the use of the learned model 231 in the present application example will be described with reference to FIG. 14. FIG. 14 is a flowchart showing an example of the information generation process according to the present application example. Since the process from step Sc1 through step Sc3 in FIG. 14 is the same the d Sb1 through step Sb3, descriptions thereof are omitted.

(Information Generation Process)

(Step Sc4)

The processing circuitry 205 outputs, via the output function 2513, pattern-related information or disease information, from the CNN 2313, to which the shift image output from the circulation shift layer 2311 and the MR image have been input. The processing circuitry 205 outputs the pattern-related information or the disease information to the memory 203 and the display 207.

(Step Sc5)

The display 207 displays the pattern-related information or the disease information output by the process in step Sc4. The processing circuitry 205 may output the pattern-related information or the disease information to an external device, such as a medical image storage device, via a network (not shown). The disease information may be stored in the memory 203 or an external storage apparatus in association with the MR image input to the learned model 231. The pattern-related information is a physical parameter, and the processing circuitry 205 may correct the MR image using the psychical parameter by an image correction function (not shown) in a subsequent step of this procedure.

If the pattern-related information is data indicative of the presence/absence of artifact, and if it is determined by the processing circuitry 205 that artifact is present in an MR image input to the learned model 231 (hereinafter, “artifact-present determination”), the processing circuitry 205 outputs an instruction to perform magnetic resonance imaging once again to a subject P (hereinafter, “re-imaging instruction”) to the present MRI apparatus 100. Specifically, the artifact-present determination serves as an impetus to prompt the processing circuitry 205 to output a re-imaging instruction to the imaging control circuitry 121. In response to the input of re-imaging instruction, the imaging-control circuitry 121 performs magnetic resonance imaging once again to a subject P.

According to the above-described configuration, the following advantageous effects can be obtained.

According to the medical signal processing apparatus 200 in the present application example, the medical signal 2033 is an MR image generated by magnetic resonance imaging performed to a subject P; the pattern is artifact caused in an MR image in accordance with an imaging condition 2031 of an MR image; the pattern-related information is a physical parameter used for correcting an MR image; the known direction is a direction relating to an occurrence of artifact; and the known shift amount is an amount of translation based on a location where artifact occurs. According to the present medical signal processing apparatus 200, the medical signal 2033 is an MR image generated by magnetic resonance imaging performed to a subject P; the pattern is artifact that occurs in an MR image in accordance with an imaging condition 2031 of an MR image; the pattern-related information is data indicating presence/absence of artifact; a known direction is a direction relating to an occurrence of artifact; and a known shift amount is an amount of translation based on a location where artifact occurs. According to the present medical signal processing apparatus 200, the medical signal 2033 is an MR image generated by magnetic resonance imaging performed to a subject P; the pattern is artifact caused in an MR image in accordance with an imaging signal 2031 of magnetic resonance imaging; the disease information is information indicating a result of identification of each of diseases in an MR image; a known direction is a direction relating to occurrence of artifact; and a known shift amount is an amount of translation based on a location where artifact occurs.

Thus, according to the present medical signal processing apparatus 200, it is possible to obtain, for an MR image which has artifact that appears at a location shifted by a known shift amount along a known direction, pattern-related information, such as at least one physical parameter used for correcting said MR image or data indicating presence/absence of artifact, and disease information, such as data indicating a result of identification of each of the diseases in the MR image. It is thereby possible to increase efficiency of diagnosis of a subject P, according to the present medical signal processing apparatus 200.

### THIRD APPLICATION EXAMPLE

A difference of the present application example from the first and second application examples lies in that a living body signal of the subject P is used as a medical signal 2033. A living body signal is a one-dimensional signal, such as an electrocardiogram waveform, a pulse waveform, and a respiration waveform. In the present application example, a pattern that appears at a location shifted by a known shift amount along a known direction corresponds to a waveform of a living body signal. A known direction is a time direction relating to the acquisition of a living body signal. A known shift amount is a period of time between predetermined time phases in a living body signal, and for example, a cycle of a living body signal.

The medical signal processing apparatus 200 may be installed in a living body signal measurement apparatus for measuring a living body signal. Hereinafter, in order to make the description specific, suppose the living body signal is an electrocardiogram waveform. In this case, the present medical signal processing apparatus 200 may be installed on an electrocardiograph, for example. In addition, the medical signal processing apparatus 200 may possess a speaker (not shown). In the description hereafter, suppose that the learned model 231 in the present application example outputs disease information.

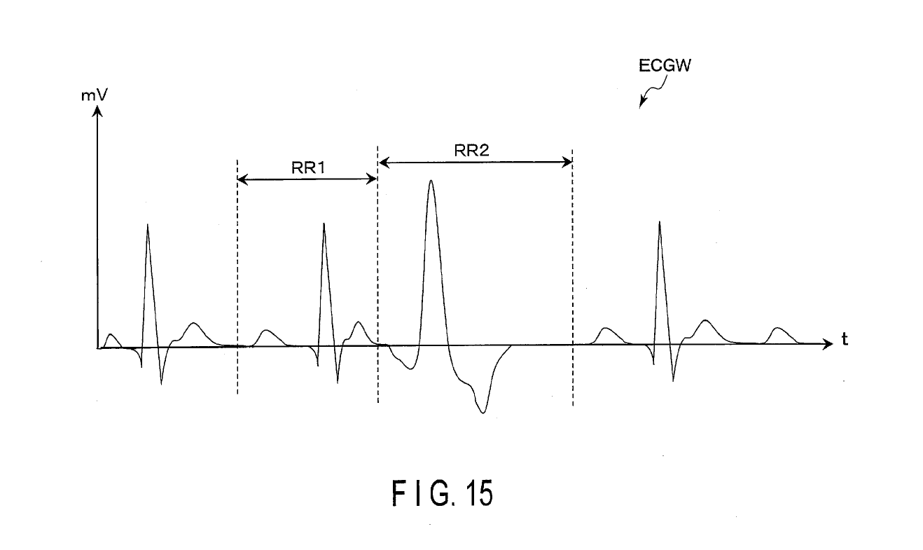

FIG. 15 is a drawing showing an example of an electrocardiogram waveform ECGW as a living body signal in the present application example. As shown in FIG. 15, a part of the electrocardiogram waveform ECGW included in R11 (hereinafter, “first waveform”) and a part of the electrocardiogram waveform ECGW included in R22 (hereinafter, “second waveform”) are input to a learned model.

The processing circuitry 205 determines a shift amount via the determination function 2511. Specifically, the processing circuitry 205 determines a shift amount based on an interval of two adjacent R waves in an electrocardiogram waveform. In the present application example, the known direction is a time direction and it becomes unnecessary to determine the known direction.

The circulation shift layer 2311 cyclically shifts a first waveform and a second waveform, for example, in accordance with the determined shift amount along the time direction. In this case, the shift signal shows the second waveform and the first waveform in the order, along the time direction. The processing circuitry 205 outputs disease information through the output function 2513 from the CNN 2313 to which the shift signal and the living body signal. The k-th output yₖ (k is a natural number) in each of the intermediate layers in the CNN 2313 can be expressed by the expression below, wherein an i-th value in an intermediate layer, or an input layer in a previous stage, is xᵢ (i is a natural number), N is a known shift amount, and w represents a weight:

$${y}_{k} = {\sum }_{i ∈ N ( k )}^{ } {w}_{k , i} {x}_{i} + {\sum }_{i ∈ N ( k + N / 2 )}^{ } {w}_{k , i} {x}_{i}$$

For example, if the electrocardiogram waveform is as shown in FIG. 15, the processing circuitry 205 outputs an index value indicating the likelihood of the disease premature ventricular contraction. The processing circuitry 205 displays a warning on the display 207 if the index value is equal to or greater than a predetermined value. In this case, the processing circuitry 205 may cause the speaker to output a warning sound.

According to the above-described configuration, the following advantageous effects can be obtained.

According to the medical signal processing apparatus 200 of the present application example, the medical signal 2033 is a living body signal of a subject P; the pattern is a waveform of a living body signal; the disease information is data indicating a result of identification of each of diseases in a living body signal; the known direction is a time direction relating to the acquisition of a living body signal; and the known shift amount is a period between predetermined time phases in a living body signal. According to the present medical signal processing apparatus 200, it is thereby possible to obtain disease information, such as data indicating a result of identification of each disease in a living body signal, which has a pattern that appears at a location shifted by a known shift amount in a known direction. It is thereby possible, according to the present medical signal processing apparatus 200, to notify an abnormal status through detection of abnormalities in a living body signal from a subject P.

### FOURTH APPLICATION EXAMPLE

A difference of the present application example from the first to third application examples lies in that a medical signal, having a pattern that appears at a location shifted by a known shift amount along a known direction, is divided based on the known direction and the known shift amount to generate a plurality of partial signals, and at least one of a corrected signal, pattern-related information, or disease information is output through an input of the plurality of partial signals into a learned model that does not have a circulation shift layer but has a DNN.

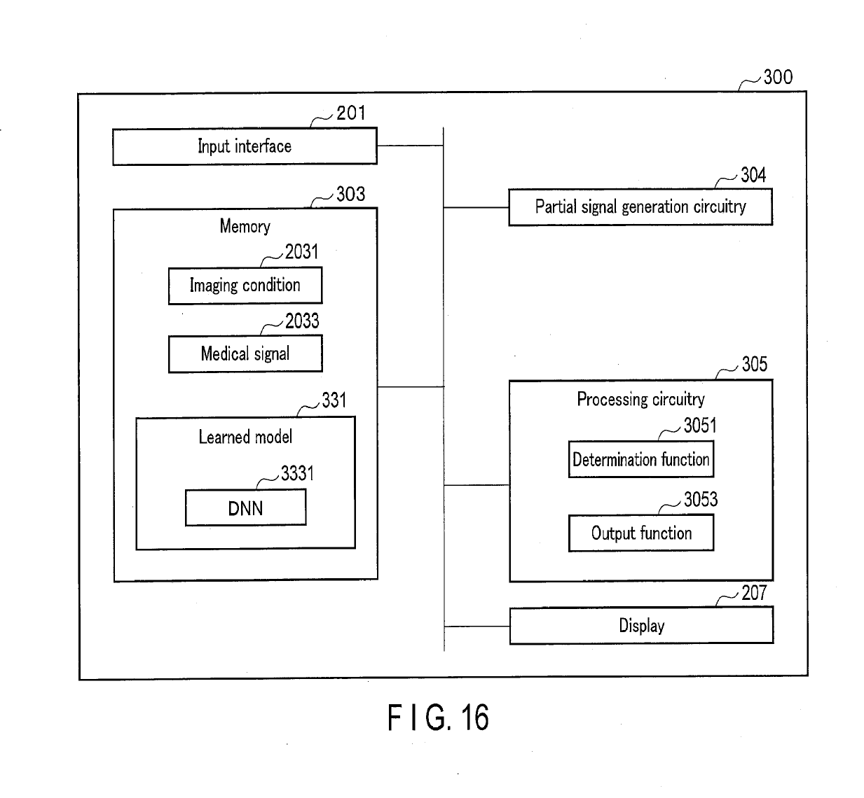

FIG. 16 is a diagram showing an example of the medical signal processing apparatus 300 according to the present application example. In the following description, the structural elements shown in FIG. 16 having substantially the same functions as the structural elements of the medical signal processing apparatus 200 shown in FIG. 10 will be denoted by the same reference symbols, and a repeat description of such elements will be given only where necessary. The medical signal processing apparatus 300 has an input interface 201, a memory 303, partial signal generation circuitry (partial signal generator) 304, processing circuitry 305, and a display 207. The partial signal generation circuitry 304 may be called an aliasing preprocessor AP, and performs preprocessing relating to aliasing (hereinafter “aliasing preprocessing”).

As the aliasing preprocessing, the partial signal generation circuitry 304 divides a medical signal based on a known direction and a known shift amount that are determined by the determination function 3051. Specifically, the partial signal generation circuitry 304 divides a medical signal in a divisional width (hereinafter, “window”) in accordance with the known shift amount along the known direction, thereby generating a plurality of partial signals corresponding to the divided medical signal. The partial signal generation circuitry 304 outputs the plurality of partial signals to the processing circuitry 305. The details of the aliasing preprocessing will be described later. The aliasing preprocessing performed by the partial signal generation circuitry 304 may be performed in the processing circuitry 305 as an aliasing preprocessing function. The partial signal generation circuitry 304 is composed of an electronic circuit, such as the above-described processor.

Windows may overlap for a medical signal. In this case, each of the partial signals has a region overlapping other partial signals (hereinafter, “overlapping region”). Inputting a plurality of partial images having an overlapping region to a learned model (DNN 3331) contributes to stability in the output of one of a corrected signal, pattern-related information, or disease information. The partial signal generation circuitry 304 may generate a plurality of partial signals, assuming that one end of a medical signal cyclically continues to the other end along a known direction, as described in the foregoing first application example.

The memory 303 stores learned models 331, imaging conditions 2031, and medical signals 2033, In more detail, the memory 303 stores a plurality of learned models which respectively correspond to a total number of windows, in turn corresponding to an imaging condition. The learned model 331 has a DNN 331. The DNN 331 may be realized by ResNet, DenseNet, U-Net, or the like. The processing pertinent to the present application example is suitable for detection and identification of a medical signal and estimation of a physical parameter, but may also be used for a purpose of denoising, as described in the first application example. In this case, the learned model 331, various types of layers are incorporated into a later stage of the DNN 331 of the learned model 331. It is thereby possible for the learned model 331 to output a corrected signal.

The processing circuitry 305 determines, through the determination function 3051, a DNN 3331 to which partial signals are input, based on the imaging condition 2031. Specifically, the processing circuitry 305 determines the DNN 3331 corresponding to a total number of windows based on a known shift amount determined by the imaging condition, in accordance with said total number of windows.

The processing circuitry 305 inputs, through the output function 3053, a plurality of partial signals respectively into a plurality of channels in the DNN 3331 determined by the determination function 3051. The processing circuitry 305 inputs the plurality of partial signals into the determined learned model, and outputs one of a corrected signal, pattern-related information, or disease information.

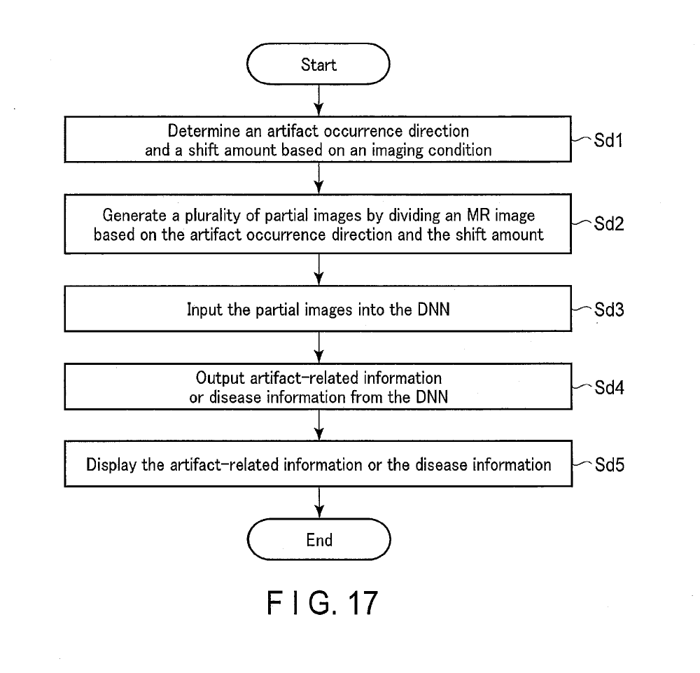

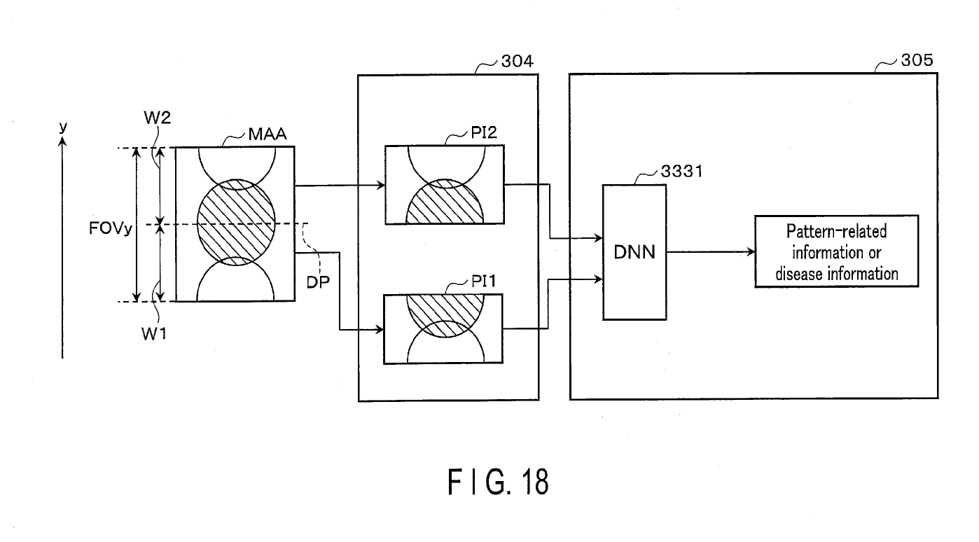

Hereinafter, to make the description specific, suppose that the medical signal is an MR image, the pattern is artifact, a partial signal is a part of the MR image (hereinafter, “partial image”), and the output from the learned model 331 is pattern-related information or disease information. A procedure of the generation of pattern-related information or disease information with the use of the learned model 331 in the present application example will be described with reference to FIGS. 17 and 18. FIG. 17 is a flowchart showing an example of a procedure of the information generation process according to the present application example. Since the processing in step Sd5 is similar to the processing in Sc5 in FIG. 14, descriptions thereof are omitted.

(Information Generation Process)

(Step Sd1)

The processing circuitry 305 determines, by the determination function 3051, an artifact occurrence direction and a shift amount by using the direction-shift amount correspondence table based on the imaging condition 2031. The processing circuitry 305 determines a DNN 3331 corresponding to a total number of windows, which corresponds to the determined shift amount.

(Step Sd2)

The partial signal generation circuitry 304 divides the MR image based on the known direction and the known shift amount to generate a plurality of partial images. Specifically, the partial signal generation circuitry 304 divides the MR signal by a window corresponding to the determined shift amount, along the artifact occurrence direction. The aliasing preprocessing for dividing an MR image will be described below with reference to FIG. 18.

FIG. 18 is a flowchart showing an example of the aliasing preprocessing in the present application example. As shown in FIG. 18, the MR image MAA to be input to the partial signal generation circuitry 304 has aliasing artifact, which is wrapped around in the phase encode direction, when the reduction factor is 2. In this case, the total number of windows used for dividing the MR image MAA is two, namely, the first window W1 and the second window W2. The partial signal generation circuitry 304 divides the MR image MAA at a location DP (hereinafter, “dividing position”) which corresponds to a half of FOVy (FOVy/2) in the phase encode direction in the MR image MAA. The partial signal generation circuitry 304 generates a first partial image PI1 and a second partial image PI2 respectively corresponding to the first window W1 and the second window W2, by dividing the MR image MAA at the dividing position DP. The partial signal generation circuitry 304 outputs the first partial image PI1 and the second partial image PI2 to the processing circuitry 305.

Each of the first window W1 and the second window W2 may be set astride the dividing position DP. For example, if FOVy in the MR image MAA is divided into ten parts, the 1/10 thorough 8/10 may be set as the first window W1, and the 6/10 through 10/10 as the second window W2, along the phase encode direction y.

(Step Sd3)

The processing circuitry 305 inputs, through the output function 3053, the plurality of partial images to the determined DNN 3331. For example, the processing circuitry 305 inputs a plurality of partial images respectively to a plurality of channels in the DNN 3331.

(Step Sd4)

The processing circuitry 305 outputs, through the output function 3053, the pattern-related information or the disease information from the DNN 3331 to which the plurality of partial images is input. The processing circuitry 305 outputs the pattern-related information or the disease information to the memory 203 and the display 207.

According to the above-described configuration, the following advantageous effects can be obtained.

According to the medical signal processing apparatus 200 of the present application example, a plurality of partial signals obtained by dividing a medical signal having a pattern appearing at a location shifted by a known shift amount along a known direction based on the known direction and the known shift amount, is generated, and said generated partial signals are input to a learned model configured to output one of a corrected signal obtained by correcting a medical signal so as to reduce a pattern, pattern-related information relating to a pattern, and disease information relating to a medical signal, thereby outputting one of said corrected signal, pattern-related information, or disease information.

### FIFTH APPLICATION EXAMPLE

A difference of the present application example from the first to fourth application examples lies in that a plurality of partial signals generated by the aliasing preprocessing are input to a DNN as a learned model, and the plurality of partial corrected signals output from a DNN are coupled based on a known direction and a known shift amount, thereby generating a coupled signal and outputting the same.

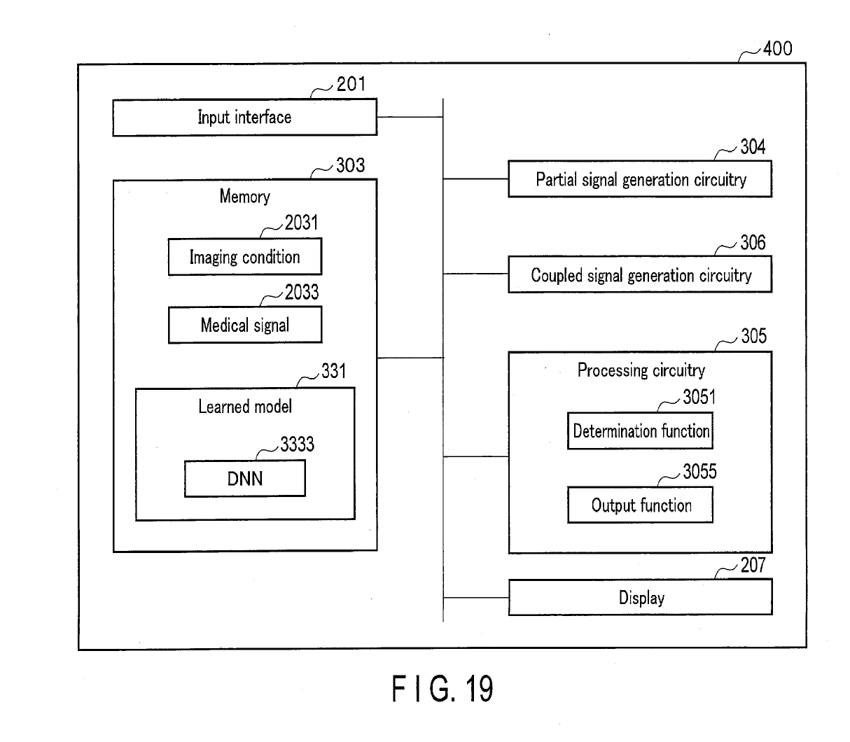

FIG. 19 is a diagram showing an example of the medical signal processing apparatus 400 according to the present application example. In the following description, the structural elements shown in FIG. 19 that have substantially the same functions as the structural elements of the medical signal processing apparatus 300 shown in FIG. 16 will be denoted by the same reference symbols, and a repeat description of such elements will be given only where necessary. The medical signal processing apparatus 400 has an input interface 201, a memory 303, partial signal generation circuitry 304, coupled signal generation circuitry 306, processing circuitry 305, and a display 207. The coupled signal generation circuitry 306 may be called an aliasing post-processor APost, and performs post-processing relating to aliasing (hereinafter “aliasing post-processing”).

According to the present application example, if an image subjected to the aliasing preprocessing is applied to the DNN 3333, an image having an original spatial resolution can be output through the performance of the aliasing post-processing. In the aliasing pre-processor AP of the fourth application example, a plurality of partial images is generated by dividing an MR image based on a known direction and a known shift amount. In contrast, the aliasing post-processor APost in the present application example, a divided MR image output from the DNN 3333 is coupled based on a known direction and a known shift amount. For example, if the aliasing preprocessor AP divides an MR image equally into three with respect to an x-axis direction, the aliasing post-processor APost couples three output images into one. When the aliasing post-processor APost is used, an image before coupling (in other words, a divided image) is used as learning data for the learning in the DNN 3333.

For example, if reconstruction is performed on a small FOV, only an image in which aliasing occurs is obtained in some cases. For the purpose of removing aliasing from such an image through the post-processing, only the aliasing post-processing may be adopted without adopting the aliasing preprocessing, assuming that the image is twice as large as a usual image with respect to the aliasing direction, for example.

The memory 303 stores learned models 331, imaging conditions 2031, and medical signals 2033. In more detail, the memory 303 stores a plurality of learned models which respectively correspond to a total number of windows, in turn corresponding to an imaging condition. The learned model 331 has a DNN 3333. The DNN 3333 may be realized by ResNet, DenseNet, U-Net, or the like. The DNN 3333 outputs a plurality of partial corrected signals respectively corresponding to a plurality of partial signals to be input to itself. The plurality of partial corrected signals corresponds to, for example, the above-described corrected signal, which is obtained by denoising the partial signals. The resolution of a medical signal input to the DNN 3333, and that of the signal output from the DNN 3333, corresponds to a resolution obtained by dividing the resolution of the medical signal 2033 with the total number of windows. Prior to the installation of DNN 3333 to the medical signal processing apparatus 400, the DNN 3333 in the present application example learns, as learning data, the plurality of partial signals corresponding to the total number of windows, and the partial corrected signal as correct data.

The processing circuitry 305 inputs, through the output function 3055, the plurality of partial signals into the learned model 331, and then outputs a plurality of corrected signals corresponding to the plurality of partial signals, as corrected signals. Specifically, the processing circuitry 305 inputs a plurality of partial signals respectively to a plurality of channels in the DNN 3331 determined by the determination function 3051. The processing circuitry 305 inputs a plurality of partial signals to the determined learned model, and outputs a plurality of partial corrected signals to the coupled signal generation circuitry 306.

The coupled signal generation circuitry 306 generates a coupled signal by coupling the plurality of partial corrected signals output from the DNN 3333, based on the known direction and the known shift amount, which are determined by the determination function 3051. The coupled signal generation circuitry 306 outputs the coupled signal to the memory 303 and the display 207. The coupled signal generation circuitry 306 is composed of an electronic circuit, such as the above-described processor. The details of the aliasing post-processing will be described later. The aliasing post-processing performed by the coupled signal generation circuitry 306 may be performed in the processing circuitry 305 as an aliasing post-processing function. In this case, the program that executes the aliasing post-processing function is stored in the memory 303, or in an ASIC of the processing circuitry 305.

Hereinafter, to make the descriptions specific, suppose that the medical signal is an MR image, the pattern is artifact, the partial signals are partial images, the partial corrected signals output from the learned model 331 are a plurality of corrected images respectively corresponding to a plurality of partial images (hereinafter, partial corrected images), and the coupled signal is an image obtained by coupling a plurality of partial corrected images (hereinafter, “coupled image”). The corrected image corresponds to the above-described artifact reduced image.

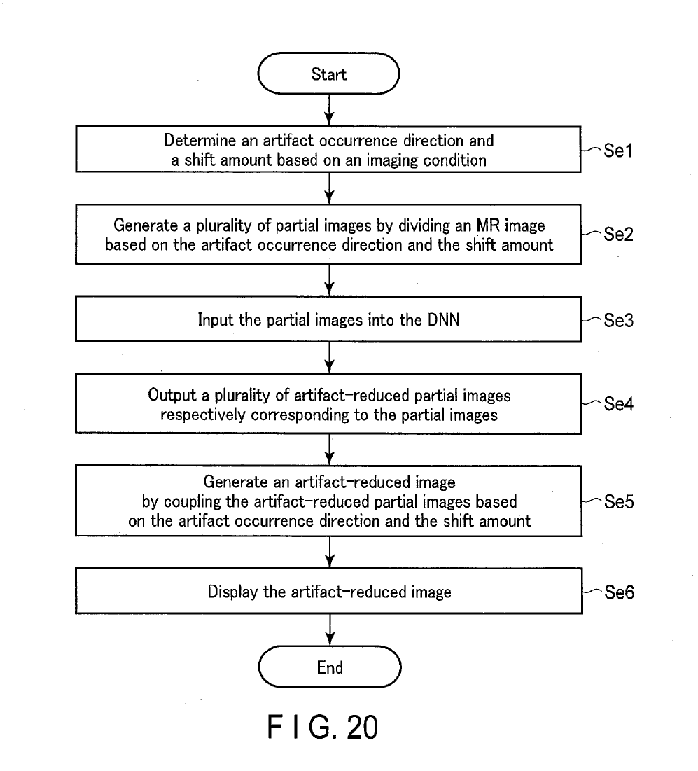

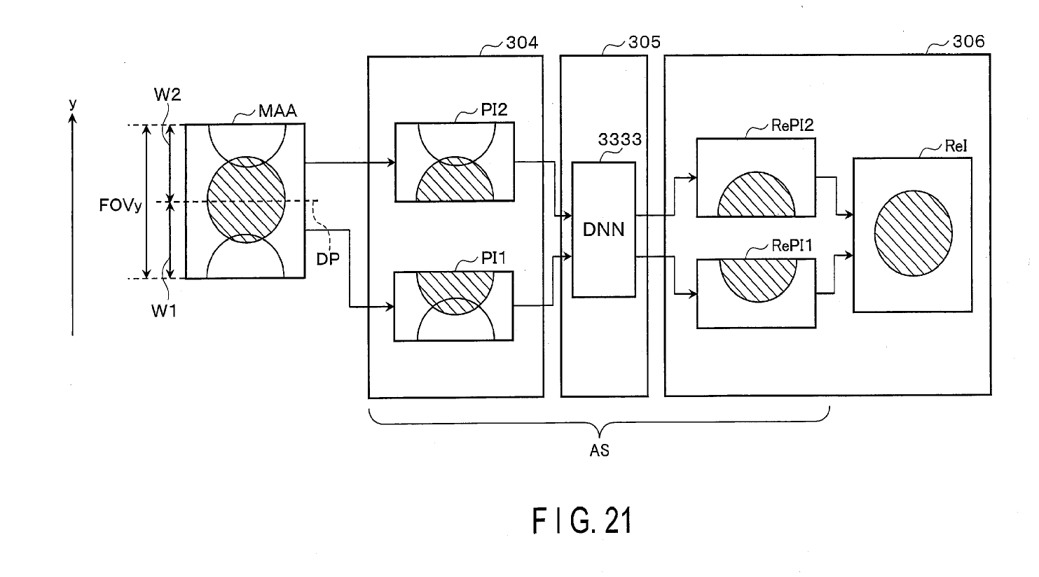

The procedure of the coupled image generation for generating a coupled image corresponding to a corrected signal in the present application example will be described below with reference to FIGS. 20 and 21. FIG. 20 is a flowchart showing an example of the coupled image generation process according to the present application example. Since the processing in step Se1 through step Se3 is similar to the processing in step Sd1 through step Sd3 in FIG. 17, descriptions thereof are omitted. Since the processing in step Se6 is similar to the processing in Sb5 in FIG. 11, descriptions thereof are omitted. In addition, in the following description, let us suppose that the aliasing preprocessor AP divides an MR image equally into two along a y-axis direction, as shown in FIG. 21.

(Coupled Image Generation Process)

(Step Se4)

The processing circuitry 305 outputs, through the output function 3055, a plurality of partial corrected images from the DNN 3333 to which the plurality of partial images is input. The partial corrected images correspond to partial images in which artifact is reduced (hereinafter, “artifact-reduced partial images”). In other words, the processing circuitry 305 outputs a plurality of artifact-reduced partial images respectively corresponding to a plurality of partial images, from the DNN 3333. The process 305 inputs a plurality of partial images into the DNN 3333, and outputs the plurality of artifact-reduced partial images (already output from the DNN 3333 ) to the coupled signal generation circuitry 306.

(Step Se5)

The coupled signal generation circuitry 306 couples the plurality of partial corrected images based on a known direction (artifact occurrence direction) and a known shift amount, which are determined by the determination function 3051. The coupled signal generation circuitry 306 generates a coupled image through this process. In other words, the coupled signal generation circuitry 306 generates an artifact-reduced image by coupling the plurality of artifact-reduced partial images based on the artifact occurrence direction and the shift amount. The coupled signal generation circuitry 306 outputs the artifact-reduced image to the memory 303 and the display 207.

FIG. 21 is an example of the aliasing post-processing in the present application example. Since the aliasing pre-processing in FIG. 21 is the same as that of FIG. 18, descriptions thereof are omitted. The processing circuitry 305 outputs, through the output function 3055, a first artifact-reduced partial image RePIl corresponding to a first partial image PI1, and a second artifact reduced partial image RePI2 corresponding to a second partial image PI2, based on the input of the first partial image PI1 and the second partial image PI2 into the DNN 3333 in the learned model 305. The coupled signal generation circuitry 306 specifies a coupled position where the first artifact-reduced partial image RePIl and the second artifact-reduced partial image RePI2 are coupled through the use of the artifact occurrence direction and the shift amount. The coupled position corresponds to, for example, the dividing position DP. Subsequently, the coupled signal generation circuitry 306 generates an artifact-reduced image by coupling the first artifact-reduced partial image RePI1 with the second artifact-reduced partial image RePI2 at the coupled position. The range indicated by “AS” in FIG. 21 shows the processing performed at half of the resolution of the MR image before being subjected to the aliasing preprocessing (hereinafter, “full resolution”). Generally, if an MR image is divided into N via the aliasing preprocessing, the processing in the range indicated by “AS” in FIG. 21 is performed at 1/N of the full resolution.

According to the above-described configuration, the following advantageous effects can be obtained.

According to the medical signal processing apparatus 400 of the present application example, a.medical signal having a pattern that appears at a location shifted by a known shift amount along a known direction is divided based on the known direction and the known shift amount to generate a plurality of partial signals, and the generated partial signals are input to a learned model configured to output a plurality of partial corrected signals which respectively correspond to the partial signals, and are corrected so as to reduce a pattern in the medical signal so that the model outputs a plurality of partial corrected signals as corrected signals, and the partial corrected signals, are coupled based on said known direction and shift amount, thereby generating a coupled signal.

As a modification of the foregoing first to third application examples, if the technical idea of the present medical signal processing apparatus 200 is realized by cloud computing or the like, a server on the Internet may have the processing circuitry 205 and the memory 203 shown in FIG. 10, for example. As a modification of the fourth embodiment, if the technical idea of the present medical signal processing apparatus 300 is realized by cloud computing or the like, a server on the Internet may have the partial signal generation circuitry 304, the processing circuitry 305, and the memory 303 shown in FIG. 16, for example. As a modification of the fifth embodiment, if the technical idea of the present medical signal processing apparatus 400 is realized by cloud computing or the like, a server on the Internet may have the partial signal generation circuitry 304, the processing circuitry 305, the coupled signal generation circuitry 306, and the memory 303 shown in FIG. 19, for example. In those cases, the system control function 2511, the determination function 3051, the output function 2513, the output function 3053, the output function 3055, etc. are realized by installing a program that executes these functions onto the processing apparatus of the server and expanding these functions in a memory.

According to at least one of the above-described embodiment and modifications, it is possible to reduce output errors stemming from a learned model. For example, according to the present medical signal processing apparatus 200, even for a medical signal having a pattern (artifact) that appears at a location shifted by a known shift amount along a known direction, it is possible to output, from an input of said medical signal to the learned model 231, at least one of a corrected signal in which reduction of noise due to artifact is improved, pattern-related information in which an identification rate is improved, or disease information; and it is also possible to increase diagnosis efficiency. According to the present medical signal processing apparatus 300, it is possible to output at least one of a corrected signal in which reduction of noise due to artifact is improved, pattern-related information in which an identification rate is improved, or disease information, through the performance of aliasing preprocessing to a medical signal having a pattern (artifact) appearing at a location shifted by a known shift amount along a known direction, and the input of the pre-processed medical signal into the learned model 331; and it is also possible to increase diagnosis efficiency. According to the present medical signal processing apparatus 400, it is possible to output a plurality of corrected signals in which reduction of noise due to artifact is improved, through the performance of the aliasing preprocessing to a medical signal having a pattern (artifact) appearing at a location shifted by a known shift amount along a known direction, and the input of the pre-processed medical signal into the learned model 331; and it is also possible to generate a coupled signal by coupling the plurality of corrected signals, and to increase diagnosis efficiency.

In any of the preprocessing, post-processing, the inside of the DNN or any combination thereof, resolution of an image may be converted via a resolution conversion process, such as upsampling, downsampling, or pooling, to output an image having a resolution different from the input image.

In an apparatus that performs the present embodiment, the availability or otherwise of a learned model may be presented to a user when the user selects an imaging method. Specifically, if a learned model is available, a function that performs the various processes in the present embodiment can be selected, and if a learned model is not available, such a function cannot be selected. Or, if a function that performs the various processes in the present embodiment is selected, an imaging condition for which a learned model is available may be selected.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical signal processing apparatus comprising:
processing circuitry that
inputs a medical signal to a learned model configured to output one of the following: a corrected signal that is corrected so as to reduce a pattern of the medical signal, the pattern appearing at a location shifted by a known shift amount in a known direction; pattern-related information relating to the pattern; or disease information relating to the medical signal, and
outputs, by using the direction and the shift amount, one of the corrected signal, the pattern-related information, or the disease information.

2. The medical signal processing apparatus according to claim 1, wherein
the learned model has
a circulation shift layer that generates a shift signal obtained by cyclically shifting the medical signal by the shift amount in the direction, and
a neural network configured to output one of the corrected signal, the pattern-related information, or the disease information by using the medical signal and the shift signal.

3. The medical signal processing apparatus according to claim 1, wherein
the medical signal is a magnetic resonance image generated by magnetic resonance imaging performed to a subject,
the pattern is artifact caused in the magnetic resonance image in accordance with an imaging condition of the magnetic resonance imaging,
the corrected signal is an artifact-reduced image in which the artifact is reduced,
the learned model is a convolutional neural network having a plurality of intermediate layers,
the processing circuitry performs processing in each of the intermediate layers so as to input a combination of an output from any first node of an intermediate layer in a preceding stage connected to an input side of each of the intermediate layers, and an output from a second node in the intermediate layer of the preceding stage, the second node being determined by the imaging condition.

4. The medical signal processing apparatus according to claim 2, wherein
the medical signal is a magnetic resonance image generated by magnetic resonance imaging performed to a subject,
the pattern is artifact that occurs in the magnetic resonance image in accordance with an imaging condition of the magnetic resonance imaging,
the corrected signal is an artifact-reduced image in which the artifact is reduced,
the neural network is a neural network having a local linear coupling in each of a plurality of intermediate layers,
the direction is a direction relating to occurrence of the artifact, and
the shift amount is an amount of translation based on the location where the artifact occurs.

5. The medical signal processing apparatus according to claim 2, wherein
the medical signal is a magnetic resonance image generated by magnetic resonance imaging performed to a subject,
the pattern is artifact caused in the magnetic resonance image in accordance with an imaging condition of the magnetic resonance imaging,
the pattern-related information is a physical parameter used for correcting the magnetic resonance image, the direction is a direction relating to occurrence of the artifact, and
the shift amount is an amount of translation based on the location where the artifact occurs.

6. The medical signal processing apparatus according to claim 2, wherein
the medical signal is a magnetic resonance image generated by magnetic resonance imaging performed to a subject,
the pattern is artifact caused in the magnetic resonance image in accordance with an imaging condition of the magnetic resonance imaging,
the pattern-related information is data indicating presence or absence of occurrence of the artifact,
the direction is a direction relating to occurrence of the artifact, and
the shift amount is an amount of translation based on the location where the artifact occurs.

7. The medical signal processing apparatus according to claim 2, wherein
the medical signal is a magnetic resonance image generated by magnetic resonance imaging performed to a subject,
the pattern is artifact caused in the magnetic resonance image in accordance with an imaging condition of the magnetic resonance imaging,
the disease information is data indicating a result of identification of each of diseases in the magnetic resonance image,
the direction is a direction relating to occurrence of the artifact, and
the shift amount is an amount of translation based on the location where the artifact occurs.

8. The medical signal processing apparatus according to claim 3, wherein
the artifact is at least one of aliasing artifact, N-half artifact, chemical shift artifact, or motion artifact.

9. The medical signal processing apparatus according to claim 2, wherein
the medical signal is a living body signal of a subject,
the pattern is a waveform of the living body signal,
the disease information is data indicating a result of identification of each disease in the living body signal,
the direction is a time direction relating to acquisition of the living body signal, and
the shift amount is a period of time within a predetermined time phase in the living body signal.

10. A medical signal processing apparatus comprising:
partial signal generation circuitry that generates a plurality of partial signals by dividing a medical signal having a pattern that appears at a location shifted by a known shift amount along a known direction, based on the direction and the shift amount; and
processing circuitry that
inputs the plurality of partial signals into a learned model configured to output one of a corrected signal that is corrected so as to reduce the pattern of the medical signal, pattern-related information relating to the pattern, or disease information relating to the medical signal, and
outputs one of the corrected signal, the pattern-related information, or the disease information.

11. The medical signal processing apparatus according to claim 10, wherein
the processing circuitry inputs the plurality of partial signals into the learned model, and outputs, as the corrected signal, a plurality of partial corrected signals respectively corresponding to the partial signals, and
the apparatus further comprises coupled signal generation circuitry that generates a coupled signal by coupling the plurality of partial corrected signals based on the direction and the shift amount.
